(19) United States
(12) Patent Application Publication
(10) Pub. No.: US 2016/0208221 A1
(43) Pub. Date: Jul. 21, 2016

(54) COMPOSITIONS AND METHODS USING CAPSIDS RESISTANT TO HYDROLASES

(71) Applicant: APSE, LLC, St. Louis, MO (US)

(72) Inventors: Juan Pedro Humberto ARHANCET, St. Louis, MO (US); Neena SUMMERS, St. Louis, MO (US); Henry HUANG, St. Louis, MO (US)

(73) Assignee: APSE, LLC, St. Louis, MO (US)

(21) Appl. No.: 14/914,553

(22) PCT Filed: Sep. 12, 2014
(86) PCT No.: PCT/US14/55426

§ 371 Date: Feb. 25, 2016

Related U.S. Application Data
(60) Provisional application No. 61/877,175, filed on Sep. 12, 2013.

(51) Int. Cl. C12N 7/00

(52) U.S. Cl. CPC C12N 7/00; C12N 2795/18123; C12N 2795/18151

(57) ABSTRACT

Novel processes and compositions are described which use viral capsid proteins resistant to hydrolases to prepare virus-like particles to enclose and subsequently isolate and purify target cargo molecules of interest including nucleic acids such as siRNAs and shRNAs, miRNAs, messenger RNAs, small peptides and bioactive molecules.

### CROSS REFERENCE TO RELATED APPLICATIONS

This application claims priority to U.S. provisional application No. 61/877,175, filed Sep. 12, 2013, the entire disclosure of which is hereby incorporated by reference.

### INCORPORATION OF SEQUENCE LISTING

The entire contents of a paper copy of the “Sequence Listing” and a computer readable form of the sequence listing on optical disk, containing the file named 462344_SequenceListing ST25.txt, which is 56 kilobytes in size and was created on Sep. 10, 2014, are herein incorporated by reference.

### TECHNICAL FIELD

The invention relates to virus-like particles, and in particular to methods and compositions using viral capsids as nanocontainers for producing, isolating and purifying heterologous nucleic acids and proteins, and delivering same to organisms.

### BACKGROUND OF THE INVENTION

Virus-like particles (VLPs) are particles derived in part from viruses through the expression of certain viral structural proteins which make up the viral envelope and/or capsid, but VLPs do not contain the viral genome and are non-infectious. VLPs have been derived for example from the Hepatitis B virus and certain other viruses, and have been used to study viral assembly and in vaccine development.

Viral capsids are composed of at least one protein, several copies of which assemble to form the capsid. In some viruses, the viral capsid is covered by the viral envelope. Such viral envelopes are comprised of viral glycoproteins and portions of the infected host's cell membranes, and shield the viral capsids from large molecules that would otherwise interact with them. The capsid is typically said to encapsidate the nucleic acids which encode the viral genome and sometimes also proteins necessary for the virus' persistence in the natural environment. For the viral genome of a virus to enter a new host, the capsid must be disassembled. Such disassembly happens under conditions normally used by the host to degrade its own as well as foreign components, and most often involves proteolysis. Viruses take advantage of normal host processes such as proteolytic degradation to enable critical part of their cycle, i.e. capsid disassembly and genome release.

It is therefore unsurprising that the research literature has not previously described capsids resistant to hydrolases that act on peptide bonds. A very limited number of certain specific peptide sequences which are part of larger proteins are known to be somewhat resistant to certain proteases, but the vast majority of peptide sequences are not. Viruses that resist proteolysis have been reported, but these are all enveloped viruses, in which the capsid is shielded by the viral envelope. In such viruses the capsids are not in contact with, i.e. they are shielded from, the proteases described. The use of such protease resistant virus capsids to produce large amounts of heterologous cargo molecules and how the protease resistant property can be exploited to facilitate purification of the heterologous cargo molecules is discussed in U.S. patent Publication No. US20130167267. In particular, Examples A through FF of U.S. patent Publication No. US20130167267 are incorporated herein by reference in their entirety.

In large-scale manufacturing of recombinant molecules such as proteins, ultrafiltration is often used to remove molecules smaller than the target protein in the purification steps leading to its isolation. Purification methods also often involve precipitation, solvent extraction, and crystallization techniques. These separation techniques are inherently simple and low cost because, in contrast to chromatography, they are not based on surface but on bulk interactions. However, these techniques are typically limited to applications to simple systems, and by the need to specify a different set of conditions for each protein and expression system. Yet each target recombinant protein presents a unique set of binding interactions, thereby making its isolation process unique and complex. The separation efficiency for recombinant proteins using these simple isolation processes is therefore low.

Nucleic acids, including siRNA and miRNA, have for the most part been manufactured using chemical synthesis methods. These methods are generally complex and high cost because of the large number of steps needed and the complexity of the reactions which predispose to technical difficulties, and the cost of the manufacturing systems. In addition, the synthetic reagents involved are costly and so economy of scale is not easily obtained by simply increasing batch size. Biosynthetic methods of manufacturing nucleic acids can, in theory, produce such molecules much more cheaply than by chemical synthesis methods. However, the lack of stability of nucleic acids and recovery of these molecules from the cells in which they are produced, often compromises any theoretical advantage biosynthesis might have. What is needed is a way of stabilizing the nucleic acids and a method for cheaply and efficiently recovering the stabilized nucleic acids from the cells that produce them. Ideally, such a method involves as few steps as possible, makes use of existing processing methodologies, recyclable materials and generates little or no waste requiring special treatment. Although U.S. Patent Publication No. US20130167267 discloses how existing protease resistant capsids may be utilized to satisfy many of these criteria, a need remains for methods to engineer specific protease sensitivity into otherwise protease resistant capsids to facilitate their removal in late stages of purification, as well as a system for identifying and modifying otherwise protease sensitive capsids to become protease resistant and thus capable of packaging larger heterologous cargo molecules. In other words, an analytical framework to make protease resistant capsids protease sensitive as well as to make protease sensitive capsids protease resistant allows use of VLPs to be extended beyond the limits of currently available protease resistant capsids.

### BRIEF SUMMARY OF THE INVENTION

In one aspect, a method for modifying a hydrolysis resistant capsid such that only a particular protease or a narrow class of proteases can hydrolyze the modified capsid, which the capsid maintains its resistance to hydrolysis by other proteases or classes of proteases. The advantage of such a capsid is that the intact VLP containing a desired heterologous cargo may be produced in vivo and purified by the methods described herein, including treatment of the cell lysate containing the VLP comprised of the modified capsids with protein hydrolases which are unable to hydrolyse the capsid. Once the VLPs are purified from the cell lysate they may be subsequently treated with a protein hydrolase which can digest the modified capsid proteins to release the heterologous cargo molecule while simultaneously digesting the capsid proteins.

In addition, the disclosure provides a method for modifying a hydrolysis sensitive capsid to become resistant to protein hydrolysis by identifying loops and surface features susceptible to particular classes of protein hydrolases. Modification of such loops or surface features to alter susceptibility to protein hydrolysates can provide VLPs suitable for packaging heterologous cargo molecules of various sizes and dimensions.

In another aspect, the present disclosure provides a composition comprising: a plurality of any of the foregoing VLPs including any of the modified capsid proteins as described herein, and one or more cell lysis products present in an amount of less than 4 grams for every 100 grams of capsid present in the composition, wherein the cell lysis products are selected from proteins, polypeptides, peptides and any combination thereof. Such a composition may comprise cell lysis products present in an amount of less than 0.5 grams, less than 0.2 grams, or less than 0.1 gram.

Any of the foregoing VLPs or compositions comprising the VLPs, the VLPs may further comprise an oligonucleotide linker coupling the heterologous cargo molecule and the viral capsid.

In another aspect, the present disclosure provides a method to purify modified viral capsids each enclosing a target cargo molecule, the method comprising: subjecting a plurality of the wild type capsids obtained from a whole cell lysate to hydrolysis using a peptide bond hydrolase category EC 3.4 which is incapable of hydrolysing the modified capsid, for a time and under conditions sufficient for at least 60, at least 70, at least 80, or at least 90 of every 100 individual polypeptides present with the capsids are cleaved, while at least 60, at least 70, at least 80, or at least 90 of every 100 capsids present before such hydrolysis remain undamaged after such hydrolysis, wherein the polypeptides are cell lysis products not enclosed in the capsids, and wherein the modified viral capsids comprise a capsid protein having a surface structure wherein any surface loops have been modified to a length of no more than 10-12 Angstroms, preferably less than 6-7 Angstroms, and/or any surface loops have a sequence which has been modified to be resistant to hydrolysis catalyzed by a peptide bond hydrolase category EC 3.4 to which it is otherwise naturally sensitive. In the method, the viral capsids can be resistant to hydrolysis catalyzed by a peptide bond hydrolase category EC 3.4, such as but not limited to peptidase K, pepsin A, papain, streptogrisin A, streptogrisin B, subtilisin and protease from Bacillus licheniformis. The method may further comprise purification of the capsids following hydrolysis, wherein purification includes at least one of a liquid-liquid extraction step, a crystallization step, a fractional precipitation step or an ultrafiltration step.

In still another aspect, the present disclosure provides a method to purify modified viral capsids each enclosing a target cargo molecule, the method comprising: subjecting a plurality of the wild type capsids obtained from a whole cell lysate to hydrolysis using a peptide bond hydrolase category EC 3.4 which is incapable of hydrolysing the modified capsid, for a time and under conditions sufficient for at least 60, at least 70, at least 80, or at least 90 of every 100 individual polypeptides present with the capsids are cleaved, while at least 60, at least 70, at least 80, or at least 90 of every 100 capsids present before such hydrolysis remain undamaged after such hydrolysis, wherein the polypeptides are cell lysis products not enclosed in the capsids, and wherein the modified viral capsids comprise a capsid protein having a surface structure wherein any surface loops have been modified to a length of no more than 10-12 Angstroms, preferably less than 6-7 Angstroms, and/or any surface loops have a sequence which has been modified to be resistant to hydrolysis catalyzed by a peptide bond hydrolase category EC 3.4 to which it is otherwise naturally sensitive. In the method, the viral capsids can be resistant to hydrolysis catalyzed by a peptide bond hydrolase category EC 3.4, such as but not limited to peptidase K, pepsin A, papain, streptogrisin A, streptogrisin B, subtilisin and protease from Bacillus licheniformis. The method may further comprise purification of the capsids following hydrolysis, wherein purification includes at least one of a liquid-liquid extraction step, a crystallization step, a fractional precipitation step or an ultrafiltration step. The VLPs may be further treated with a peptide bond hydrolase category EC 3.4 to which it is otherwise naturally resistant to digest the capsid protein and facilitate further purification the heterologous cargo molecule.

### BRIEF DESCRIPTION OF THE DRAWINGS

FIG. 1 is an alignment of complete leviviridae viral coat protein sequences retrieved from the Uniprot database and aligned using the BLAST multiple alignment tool with default values for weighting array choice, gap penalties, etc.

FIG. 2 is a graphic illustration of the backbone superposition of 1AQ3 chain B (leviviridae coat protein monomer) with 1 QBE chain C (alleoviridae coat protein monomer).

FIG. 3 is a graphic illustration of an alternative view of the backbone superposition of A1Q3 chain B (leviviridae coat protein monomer) with 1QBE chain C (alleoviridae coat protein monomer) shown in FIG. 2.

FIG. 4 is a graphic illustration of another alternative view of the backbone superposition of A1Q3 chain B (leviviridae coat protein monomer) with 1QBE chain C (alleoviridae coat protein monomer) shown in FIG. 2.

FIG. 5 is a graphic illustration of another alternative view of the backbone superposition of A1Q3 chain B (leviviridae coat protein monomer) with 1QBE chain C (alleoviridae coat protein monomer) shown in FIG. 2.

FIG. 6 is a structural sequence alignment of 1AQ3, 2VTU and 1 QBE using jFATCAT rigid.

FIG. 7 is an alignment of complete alloleviviridae viral coat protein sequences retrieved from the UniProt database and aligned using the BLAST multiple alignment with default values for weighting array choice, gap penalties, etc.

FIG. 8 is a graphic illustration showing 60 of the 180 monomers forming the icosahedral levi- and alloleviviridae capsid. The backbone of each monomer is represented by a ribbon of a different shade. Backbone hydrogen bonds are represented by darker lines. The icosahedral three-fold axis is in the center of the figure. Monomer-monomer contacts do not fill the central circle outlined by hydrogen bonds connecting the tips of flexible loops 67-81.

FIG. 9 is a graphic illustration showing 2 MS2 monomers (ribbons representing backbone shaded dark and light) surrounded by monomers in contact in the icosahedral capsid (ribbons representing monomer backbones in contrasting shades). The alloleviviridae Qbeta has a two residue deletion with respect to leviviridae between 72 and 73 (red, bottom center). The central void is immediately below this deletion site. The deletion causes it to slightly expand. The Qbeta deletion at 126 (indicated, central left) removes the excursion from the segment but extensive contacts between the sheets of neighboring monomers essentially holds the monomers in place. MS2 sequence numbering is used.

FIG. 10 is a graphic illustration showing 2 MS2 monomers (ribbons representing backbone shaded dark and light) surrounded by monomers in contact in the icosahedral capsid (ribbons representing monomer backbones in contrasting shades). The alloleviviridae Qbeta has a one residue insertion with respect to leviviridae between residues 12 and 13 (yellow, top left center), a flexible loop that extends from the outer capsid surface into solvent; a two residue insertion between residues 53 and 54 (lighter segment, lower left central) at the end of a strand connection extending into the interior cargo space of the assembled capsid; a one-residue insertion between residues 27 and 28 is also at the end of a beta-strand connector extending into the capsid cargo space. None of these insertions require movement in the monomer fold or between neighbors.

FIG. 11 is a graphic illustration showing 2 MS2 monomers (ribbons representing backbone shaded dark and light) surrounded by monomers in contact in the icosahedral capsid (ribbons representing monomer backbones in contrasting shades). The alloleviviridae Qbeta has a one residue insertion with respect to leviviridae between residues 36 and 37 (lighter segment, center right). The loop packs against the end of the adjacent helix but inserted residues can extend into the central space above the flexible loop immediately below.

FIG. 12 is a graphic illustration of backbone ribbons of 3 noncovalent Enterobacteria phage MS2 noncovalent dimers packed around a symmetry point in the assembled capsid, with all of the N-termini shaded dark, the C-termini shaded light.

FIG. 13 is a series of space filling models of representative examples of VLP surface texture.

FIG. 14 is a diagram of domain folds characteristic of SCOP structure class RNA bacteriophage capsid protein (left) and nucleoplasmin-like/VP (viral coat and capsid proteins) (right).

FIG. 15 is a backbone ribbon diagram of a portion of the surface of a leviviridae MS2 capsid reconstructed from PDB-ID: 1AQ3. Capsid protein is displayed as a white ribbon, whereas fragments of encapsulated RNA localized in the electron density are displayed in darker shade.

FIGS. 16 a-g are a series of backbone ribbon diagrams of portions of selected viral capsid proteins used for VLPs. In each figure the individual asymmetric units are given their own shade. FIG. 16a depicts a portion of the black beetle virus, a T=3 alphanodavirus capsid (PDB-ID:2BBV). A single asymmetric unit is displayed on the left with each capsid protein shown as a contrasting shade backbone ribbons for clarity. The bases of localized RNA are shown as darker plates. FIG. 16b depicts a portion of the tomato aspermy virus, a T=3 bromovirus capsid (PDB-ID:2BBV). FIG. 16c depicts a portion of the satellite tobacco necrosis virus, a T=1 satellite virus capsid (PDB-ID:2BUK). FIG. 16d depicts a portion of the physalis mottle virus, a T=3 tymovirus capsid (PDB-ID:1E57). FIG. 16e depicts a portion of the tomato bushy stunt virus, a T=3 tombusvirus capsid (PDB-ID:2TBV). FIG. 16f depicts a portion of the infectious bursal disease virus, a T=1 satellite virus capsid (PDB-ID:2DF7). FIG. 16g depicts a portion of the bacteriophage phi-X174 virus, a T=1 microvirus capsid (PDB-ID:2BUK).

FIG. 17 is a schematic illustration of the outside of the leviviridae MS2 capsid a T=3 icosahedral capsid. There are 60 asymmetric units in an icosahedral capsid. Each solid numbered triangle represents one asymmetric unit. In a T=3 capsid, each asymmetric unit is comprised of 3 capsid proteins. The tips of the loops of the 3 MS2 capsid proteins (PDB-ID:1AQ3) in the asymmetric unit are connected by the dashed black lines. Representative distances (in Angstroms) between loop tips are shown in dark lines (within the asymmetric unit) and lighter lines (between asymmetric units).

FIGS. 18 a-c are a series of schematic illustrations of protein x-ray structure PDB-ID:1YU6, subtilisin A from Bacillus licheniformis complexed with the Kazal domain protein OMTKY3. The backbone ribbon diagram of subtilisin is gray, the OMTYK3 ribbon is darker. OMTYK3 residues which form backbone hydrogen bonds with subtilisin are denoted with a contrasting segment of ribbon. The complex is oriented as described in the Enzyme section of Example B with the magenta ribbon generally along the x-axis and a section of the translated x-y plane shown approximately edge-on in white. OMTK3 residues above the plane penetrate subtilisin binding cleft in order to interact with its active site. The plane as shown is translated down along the z-axis without rotation to emphasize the volume of the enzyme that must be accommodated by the local topology of the substrate if encounters are to be productive. Residues participating in hydrogen bonds are shown explicitly. Nitrogen atoms are darker, oxygen lighter, sulfur pale white, and hydrogen are white. The hydrogen bonds listed in Table 8 are shown in orange. FIG. 18a shows one subtilisin:OMTKY3 complex. FIG. 18b shows the enzyme binding cleft in close-up. FIG. 18c is an alternative view of the binding cleft rotated approximately 90 degrees with respect to FIG. 18b.

### DETAILED DESCRIPTION OF THE INVENTION

Section headings as used in this section and the entire disclosure herein are not intended to be limiting. All patents and publications cited herein are herein incorporated by reference in their entirety.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The use of “or” means “and/or” unless stated otherwise. Furthermore, the use of the term “including”, as well as other forms, such as “includes” and “included”, is not limiting.

Unless otherwise defined herein, scientific and technical terms used in connection with the present disclosure shall have the meanings that are commonly understood by those of ordinary skill in the art. For example, any nomenclatures used in connection with, and techniques of, animal and cellular anatomy, cell and tissue culture, biochemistry, molecular biology, immunology, and microbiology described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

A wide variety of conventional techniques and tools in chemistry, biochemistry, molecular biology, and immunology are employed and available for practicing the methods and compositions described herein, are within the capabilities of a person of ordinary skill in the art and well described in the literature. Such techniques and tools include those for generating recombinant capsid proteins, including capsids containing point mutations as well as insertional and deletional mutations, as well as generating and purifying VLPs including those with a wild type or a recombinant capsid together with the cargo molecule(s), and for transforming host organisms and expressing recombinant proteins and nucleic acids as described herein. See, e.g., MOLECULAR CLONING, A LABORATORY MANUAL 2^{nd} ed. 1989 (Sambrook et al., Cold Spring Harbor Laboratory Press); and CURRENT PROTOCOLS IN MOLECULAR BIOLOGY (Eds. Ausubel et al., Greene Publ. Assoc., Wiley-Interscience, NY) 1995. The disclosures in each of these are herein incorporated by reference.

As used herein, the term “cargo molecule” refers to an oligonucleotide, polypeptide or peptide molecule, which is or may be enclosed by a capsid.

An oligonucleotide may be an oligodeoxyribonucleotide (DNA) or a oligoribonucleotide (RNA), and encompasses RNA molecules such as, but not limited to, siRNA, shRNA, sshRNA, miRNA and mRNA. Certain RNA molecules may also be referred to as “active RNAs” a term meant to denote any RNA with a functional activity, including RNAi, ribozyme or packing activities.

As used herein, the term “peptide” refers to a polymeric molecule which minimally includes at least two amino acid monomers linked by peptide bond, and preferably has at least about 10, and more preferably at least about 20 amino acid monomers, and no more than about 60 amino acid monomers, preferably no more than about 50 amino acid monomers linked by peptide bonds. For example, the term encompasses polymers having about 10, about 20, about 30, about 40, about 50, or about 60 amino acid residues.

As used herein, the term “polypeptide” refers to a polymeric molecule including at least one chain of amino acid monomers linked by peptide bonds, wherein the chain includes at least about 70 amino acid residues, preferably at least about 80, more preferably at least about 90, and still more preferably at least about 100 amino acid residues. As used herein the term encompasses proteins, which may include one or more linked polypeptide chains, which may or may not be further bound to cofactors or other proteins. The term “protein” as used herein is used interchangeably with the term “polypeptide.”

As used herein, the term “variant” with reference to a molecule is a sequence that is substantially similar to the sequence of a native or wild type molecule. With respect to nucleotide sequences, variants include those sequences that may vary as to one or more bases, but because of the degeneracy of the genetic code, still encode the identical amino acid sequence of the native protein. Variants include naturally occurring alleles, and nucleotide sequences which are engineered using well-known techniques in molecular biology, such as for example site-directed mutagenesis, and which encode the native protein, as well as those that encode a polypeptide having amino acid substitutions. Generally, nucleotide sequence variants of the invention have at least 40%, at least 50%, at least 60%, at least 70% or at least 80% sequence identity to the native (endogenous) nucleotide sequence. The present disclosure also encompasses nucleotide sequence variants having at least about 85% sequence identity, at least about 90% sequence identity, at least about 85%, 86%, 87%, 88%, 89%, 90% 91%, 92%, 93%, 94%, 95%, 96%, 97%, 98%, 99%.

Sequence identity of amino acid sequences or nucleotide sequences, within defined regions of the molecule or across the full-length sequence, can be readily determined using conventional tools and methods known in the art and as described herein. For example, the degree of sequence identity of two amino acid sequences, or two nucleotide sequences, is readily determined using alignment tools such as the NCBI Basic Local Alignment Search Tool (BLAST) (Altschul, et al., 1990), which are readily available from multiple online sources. Algorithms for optimal sequence alignment are well known and described in the art, including for example in Smith and Waterman, Adv. Appl. Math. 2:482 (1981); Pearson and Lipman Proc. Natl. Acad. Sci. (U.S.A.) 85: 2444 (1988). Algorithms for sequence analysis are also readily available in programs such as blastp, blastn, blastx, tblastn and tblastx. For the purposes of the present disclosure, two nucleotide sequences may be also considered “substantially identical” when they hybridize to each other under stringent conditions. Stringent conditions include high hybridization temperature and low salt hybridization buffers which permit hybridization only between nucleic acid sequences that are highly similar. Stringent conditions are sequence-dependent and will be different in different circumstance, but typically include a temperature at least about 60°, which is about 10° C. to about 15° C. lower than the thermal melting point (Tm) for the specific sequence at a defined ionic strength and pH. Salt concentration is typically about 0.02 molar at pH 7.

As used herein with respect to a given nucleotide sequence, the term “conservative variant” refers to a nucleotide sequence that encodes an identical or essentially identical amino acid sequence as that of a reference sequence. Due to the degeneracy of the genetic code, whereby almost always more than one codon may code for each amino acid, nucleotide sequences encoding very closely related proteins may not share a high level of sequence identity. Moreover, different organisms have preferred codons for many amino acids, and different organisms or even different strains of the same organism, e.g., E. coli strains, can have different preferred codons for the same amino acid. Thus, a first nucleotide acid sequence which encodes essentially the same polypeptide as a second nucleotide acid sequence is considered substantially identical to the second nucleotide sequence, even if they do not share a minimum percentage sequence identity, or would not hybridize to one another under stringent conditions. Additionally, it should be understood that with the limited exception of ATG, which is usually the sole codon for methionine, any sequence can be modified to yield a functionally identical molecule by standard techniques, and such modifications are encompassed by the present disclosure. As described herein below, the present disclosure specifically contemplates protein variants of a native protein, which have amino acid sequences having at least 15%, at least 16%, at least 21%, at least 40%, at least 41%, at least 52%, at least 53%, at least 56%, at least 59% or at least 86% sequence identity to a native nucleotide sequence.

The degree of sequence identity between two amino acid sequences may be determined using the BLASTp algorithm of Karlin and Altschul (Proc. Natl. Acad. Sci. USA 87:2264-2268, 1993). The percentage of sequence identity is determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which an identical amino acid occurs in both sequences to yield the number of matched positions, dividing the number of matched positions by the total number of positions in the window of comparison and multiplying the result by 100 to yield the percentage of sequence identity.

One of skill will recognize that polypeptides may be “substantially similar” in that an amino acid may be substituted with a similar amino acid residue without affecting the function of the mature protein. Polypeptide sequences which are “substantially similar” share sequences as noted above except that residue positions, which are not identical, may have conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine.

A nucleic acid encoding a peptide, polypeptide or protein may be obtained by screening selected cDNA or genomic libraries using a deduced amino acid sequence for a given protein. Conventional procedures using primer extension procedures, as described for example in Sambrook et al., can be used to detect precursors and processing intermediates.

### B. VLPs Composed of a Capsid Enclosing a Cargo Molecule

The methods and compositions described herein are the result in part of the appreciation that certain viral capsids can be prepared and/or used in novel manufacturing and purification methods to improve commercialization procedures for nucleic acids. The methods described herein use recombinant viral capsids which are resistant to readily available hydrolases, to enclose heterologous cargo molecules such as nucleic acids, peptides, or polypeptides including proteins.

The capsid may be a wild type capsid or a mutant capsid derived from a wild type capsid, provided that the capsid exhibits resistance to hydrolysis catalyzed by at least one hydrolase acting on peptide bonds when the capsids are contacted with the hydrolase. Furthermore, such capsids may be modified to allow hydrolysis by at least one hydrolase acting on peptide bonds to which the capsid is otherwise resistant. As used interchangeably herein, the phrases “resistance to hydrolysis” and “hydrolase resistant” refer to any capsid which, when present in a whole cell lysate also containing polypeptides which are cell lysis products and not enclosed or incorporated in the capsids, and subjected to hydrolysis using a peptide bond hydrolase category EC 3.4 for a time and under conditions sufficient for at least 60, at least 70, at least 80, or at least 90 of every 100 individual polypeptides present in the lysate (which are cell lysis products and not enclosed in the capsids) to be cleaved (i.e. at least 60%, at least 70%, at least 80%, or at least 90% of all individual unenclosed polypeptides are cleaved), yet at least 60, at least 70, at least 80, or at least 90 of every 100 capsids present before such hydrolysis remain intact following the hydrolysis. Hydrolysis may be conducted for a period of time and under conditions sufficient for the average molecular weight of cell proteins remaining from the cell line following hydrolysis is less than about two thirds, less than about one half, less than about one third, less than about one fourth, or less than about one fifth, of the average molecular weight of the cell proteins before the hydrolysis is conducted. Methods may further comprise purifying the intact capsid remaining after hydrolysis, and measuring the weight of capsids and the weight of total dry cell matter before and after hydrolysis and purification, wherein the weight of capsids divided by the weight of total dry cell matter after hydrolysis and purification is at least twice the weight of capsids divided by the weight of total dry cell matter measured before the hydrolysis and purification. The weight of capsids divided by the weight of total dry cell matter after hydrolysis and purification may be at least 10 times more than, preferably 100 times more than, more preferably 1,000 times more than, and most preferably 10,000 times more than the weight of capsids divided by the weight of total dry cell matter measured before such hydrolysis and purification.

Hydrolases are enzymes that catalyze hydrolysis reactions classified under the identity number E.C. 3 by the Enzyme Commission. For example, enzymes that catalyze hydrolysis of ester bonds have identity numbers starting with E.C. 3.1. Enzymes that catalyze hydrolysis of glycosidic bonds have identity numbers starting with E.C. 3.2. Enzymes that catalyze hydrolysis of peptide bonds have identity numbers starting with E.C. 3.4. Proteases, which are enzymes that catalyze hydrolysis of proteins, are classified using identity numbers starting with E.C. 3.4, including but not limited to Proteinase K and subtilisin. For example, Proteinase K has identity number E.C. 3.4.21.64. The present disclosure encompasses VLPs which are resistant, in non-limiting example, Proteinase K, Protease from Streptomyces griseus, Protease from Bacillus licheniformis, pepsin and papain, and methods and processes of using such VLPs.

The Nomenclature Committee of the International Union of Biochemistry and Molecular Biology (IUBMB) also recommends naming and classification of enzymes by the reactions they catalyze. Their complete recommendations are freely and widely available, and for example can be accessed online at http://enzyme.expasy.org and, www.chem.qmul.ac.uk/iubmb/enzyme/, among others. The IUBMB developed shorthand for describing what sites each enzyme is active against. Enzymes that indiscriminately cut are referred to as broadly specific. Some enzymes have more extensive binding requirements so the description can become more complicated. For an enzyme that catalyzes a very specific reaction, for example an enzyme that processes prothrombin to active thrombin, then that activity is the basis of the cleavage description. In certain instances the precise activity of an enzyme may not be clear, and in such cases, cleavage results against standard test proteins like B-chain insulin are reported.

The capsids can be further selected and/or prepared such that they can be isolated and purified using a simple isolation and purification procedures, as described in further detail herein. For example, the capsids can be selected or genetically modified to have significantly higher hydrophobicity than a surrounding matrix as described herein, so as to selectively partition into a non-polar water-immiscible phase into which they are simply extracted. Alternatively, a capsid may be selected of genetically modified for improved ability to selectively crystallize from solution.

Use of simple and effective purification processes using the capsids is enabled by the choice of certain wild type capsids, or modifications to the amino acid sequence of proteins comprising the wild type capsids, such that the capsid exhibits resistance to hydrolysis catalyzed by at least one hydrolase acting on peptide bonds as described herein above. Methods and compositions for effecting such purifications are described in Examples A through FF of U.S. Patent Publication No. 20130167267 and are incorporated herein. The present disclosure encompasses a composition differing from those described in U.S. 20130167267 in that the capsids may be modified to become nonresistant to at least one peptide hydrolase to which the VLPs they comprise are otherwise resistant, or conversely, the capsids may be modified to be resistant to at least one peptide hydrolase to which the VLPs they comprise are otherwise nonresistant. The disclosure includes compositions of such capsids comprising: a) a plurality of VLPs each comprising a wild type viral capsid and at least one target heterologous cargo molecule enclosed in the wild type viral capsid; and b) one or more cell lysis products present in an amount of less than 40 grams, less than 30 grams, less than 20 grams, less than 15 grams, less than 10 grams, and preferably less than 9, 8, 7, 6, 5, 4, 3, more preferably less than 2 grams, and still more preferably less than 1 gram, for every 100 grams of capsid present in the composition, wherein the cell lysis products are selected from proteins, polypeptides, peptides and any combination thereof. Subsequently the cargo molecules can be readily harvested from the capsids. Accordingly, such compositions are highly desirable for all applications where high purity and/or high production efficiency is required.

VLPs as described herein may be used to enclose different types of cargo molecules to form a VLP. The cargo molecule can be but is not limited to any one or more oligonucleotide or oligoribonucleotide (DNA, RNA, LNA, PNA, siRNA, shRNA, sshRNA, lshRNA, miRNA or mRNA, or any oligonucleotide comprising any type of non-naturally occurring nucleic acid), any peptide, polypeptide or protein. A cargo molecule which is an oligonucleotide or oligoribonucleotide may be enclosed in a capsid with or without the use of a linker. A capsid can be triggered for example to self-assemble from capsid protein in the presence of nucleotide cargo, such as an oligoribonucleotide. In non-limiting example, a capsid as described herein may enclose a target heterologous RNA strand, such as for example a target heterologous RNA strand containing a total of between 1,800 and 2,248 ribonucleotides, including the 19-mer packing sequence from Enterobacteria phage MS2, such RNA strand transcribed from a plasmid separate from a plasmid coding for the capsid proteins, as described by Wei, Y., et al., (2008) J. Clin. Microbiol. 46:1734-1740.

Purification of capsids, VLPs or proteins may also include methods generally known in the art. For example, following capsid expression and cell lysis, the resulting lysate can be subjected to one or more isolation or purification steps. Such steps may include for example enzymatic lipolysis, DNA hydrolysis, and proteolysis steps. A proteolysis step may be performed for example using a blend of endo- and exo-proteases. For example, after cell lysis and hydrolytic disassembly of most cell components, such capsids with their cargo molecules can be separated from surrounding matrix by extraction, for example into a suitable non-polar water-immiscible solvent, or by crystallization from a suitable solvent. For example, hydrolysis and/or proteolysis steps transform contaminants from the capsid that are contained in the lysate matrix into small, water soluble molecules. Hydrophobic capsids may then be extracted into an organic phase such as 1, 3-bis(trifluoromethyl)benzene. Purification of capsids, VLPs or proteins may include for example at least one liquid-liquid extraction step, at least one fractional precipitation step, at least one ultrafiltration step, or at least one crystallization step. A liquid-liquid extraction may comprise for example use of an immiscible non-aqueous non-polar solvent, such as but not limited to benzene, toluene, hexane, heptane, octane, chloroform, dichloromethane, or carbon tetrachloride. Purifying may include at least one crystallization step. Use of one or more hydrolytic steps, and especially of one or more proteolytic steps, eliminates certain problems observed with current separation processes used for cargo molecules, which are mainly result from the large number and varying degree of binding interactions which take place between cargo molecules and components derived from the cell culture in which they are produced. The capsids described herein resist hydrolytic steps such that the matrix which results after hydrolysis includes intact capsids which safely partition any cargo molecules from the surrounding matrix, thereby interrupting the troublesome binding interactions which interfere with current purification processes.

Following purification, the capsid can be opened to obtain the cargo molecule, which maybe a protein or polypeptide, a peptide, or a nucleic acid molecule as described in US Patent Publication No. 20130167267, incorporated herein. Capsids can be opened using any one of several possible procedures known in the art, including for example heating in an aqueous solution above 50° C.; repeated freeze-thawing; incubating with denaturing agents such as formamide; by incubating with one or more proteases; or by a combination of any of these procedures. Capsid proteins no longer assembled in VLPs can then be removed by treatment with protein hydrolases to which they are not resistant, further facilitating purification of protease resistant heterologous cargo molecules.

Capsid proteins which are resistant to hydrolases and useful in the VLPs and methods according to the present disclosure can also be variants of, or derived from the wild type MS2 capsid protein. Capsid proteins may comprise, for example, at least one substitution, deletion or insertion of an amino acid residue relative to the wild type MS2 capsid amino acid sequence. Such capsid proteins may be naturally occurring variants or can be obtained by genetically modifying the MS2 capsid protein using conventional techniques, provided that the variant or modified capsid protein forms a non-enveloped capsid which is resistant to hydolysis catalyzed by a peptide bond hydrolases. Further, such capsid proteins may be genetically modified such that non-enveloped capsids which are resistant to hydrolysis by a specific peptide bond hydrolase or group of peptide bond hydrolases are not resistant to other peptide bond hydrolases allowing differential hydrolysis of the capsids at different stages of purification. Likewise, capsid proteins which are not resistant to hydrolysis by a specific peptide bond hydrolase or group of peptide bond hydrolases may be genetically modified to become resistant to a specific peptide bond hydrolases or group of peptide bond hydrolases allowing differential hydrolysis of the capsids at different stages of purification. This has the added benefit of allowing use of capsids forming VLPs that would otherwise not be useful for peptide hydrolase based purification of heterologous cargo molecules.

Genetically modified capsid proteins which can assemble into capsids which are resistant to hydrolysis as described herein can be engineered by making select modifications in the amino acid sequence according to conventional and well-known principles in physical chemistry and biochemistry to produce a protein which retains resistance to hydrolysis as described herein and in the Examples herein below.

It is common knowledge for example that the shape or global fold of a functional protein is determined by the amino acid sequence of the protein, and that the fold defines the protein's function. The global fold is comprised of one or more folding domains. When more than one folding domain exists in the global fold, the domains generally bind together, loosely or tightly along a domain interface. The domain fold can be broken down into a folding core of tightly packed, well-defined secondary structure elements which is primarily responsible for the domain's shape and a more mobile outer layer typically comprised of turns and loops whose conformations are influenced by interactions with the folding core as well as interactions with nearby domains and other molecules, including solvent and other proteins. An extensive public domain database of protein folds, the Structural Classification of Proteins (SCOP) database (Alexey G Murzin, Curr Opin Struct Biol (1996) 6, 386-394) of solved protein structures in the public domain is maintained online at http://scop.berkeley.edu and regularly expanded as new solved structures enter the public domain (Protein Data Bank (F. C. Bernstein, T. F. Koetzle, G. J. Williams, E. E. Meyer Jr., M. D. Brice, J. R. Rodgers, O. Kennard, T. Shimanouchi, M. Tasumi, “The Protein Data Bank: A Computer-based Archival File For Macromolecular Structures,” J. of. Mol. Biol., 112 (1977): 535), http://www.rcsb.org) database. Members of a family which are evolutionarily distant, yet have the same shape and very similar function, commonly retain as few as 30% identical residues at topologically and/or functionally equivalent positions. In some families, sequences of distant members have as few as 20% of their residues unchanged with respect to each other, e.g. levi- and alloleviviridae capsid proteins. Further, the fold and function of a protein is remarkably tolerant to change via directed or random mutation, even of core residues (Peter O. Olins, S. Christopher Bauer, Sarah Braford-Goldberg, Kris Sterbenz, Joseph O. Polazzi, Maire H. Caparon, Barbara K. Klein, Alan M. Easton, Kumnan Paik, Jon A. Klover, Barrett R. Thiele, and John P. McKearn (1995) J Biol Chem 270, 23754-23760; Yiqing Feng, Barbara K. Klein and Charles A. McWherter (1996), J Mol Biol 259, 524-541; Dale Rennell, Suzanne E. Bouvier, Larry W. Hardy and Anthony R. Poteetel (1991) J Mol Biol 222, 67-87), insertion/deletion of one or more residues (Yiqing Feng, Barbara K. Klein and Charles A. McWherter (1996), J Mol Biol 259, 524-541), permutation of the sequence (Multi-functional chimeric hematopoietic fusion proteins between sequence rearranged c-mpl receptor agonists and other hematopoietic factors, U.S. Pat. No. 6,066,318), concatenation via the N- or C-terminus or both (to copies of itself or other peptides or proteins) (Multi-functional chimeric hematopoietic fusion proteins between sequence rearranged g-csf receptor agonists and other hematopoietic factors, US20040171115; Plevka, P., Tars, K., Liljas, L. (2008) Protein Sci. 17: 173) or covalent modification, e.g., glycosylation, pegylation, SUMOylation or the addition of peptidyl or nonpeptidyl affinity tags as long as the residues critical to maintaining the fold and/or function are spared.

VLPs according to the present disclosure and as used in any of the methods and processes, thus encompass those comprising a capsid protein having at least 15%, 16%, 21%, 40%, 41%, 52%, 53%, 56%, 59% or at least 86% sequence identity with the amino acid sequence of wild type Enterobacteria phage MS2 capsid protein (SEQ ID NO: 1). Such VLPs include for example a VLP comprising a capsid protein having at least 52% sequence identity with SEQ ID NO: 1) as described above. Also included is a VLP comprising a capsid protein having at least 53% sequence identity to SEQ ID NO: 1, which can be obtained substantially as described above but not disregarding the FR capsid sequence, representing 53% sequence identity to wild-type enterobacteria phage MS2 capsid protein (SEQ ID NO: 1). Also included is a VLP comprising a capsid protein having at least 56% sequence identity to SEQ ID NO: 1, when it is considered that when the structures identified as 1AQ3 (van den Worm, S. H., Stonehouse, N.J., Valegard, K., Murray, J. B., Walton, C., Fridborg, K., Stockley, P. G., Liljas, L. (1998) Nucleic Acids Res. 26: 1345-1351) (SEQ ID NO: 2), 1GAV (Tars, K., Bundule, M., Fridborg, K., Liljas, L. (1997) J.Mol.Biol. 271: 759-773) (SEQ ID NO: 3), 1FRS (Liljas, L., Fridborg, K., Valegard, K., Bundule, M., Pumpens, P. (1994) J.Mol.Biol. 244: 279-290) (SEQ ID NO: 4) and 2VTU (Plevka, P., Tars, K., Liljas, L. (2008) Protein Sci. 17: 1731) (SEQ ID NO: 5), only 56% of the sequence positions have identical sequence and topologically equivalent positions with respect to the backbone overlays when all three sequences are considered together. Also included is a VLP comprising a capsid protein having at least 59% sequence identity to SEQ ID NO: 1, when it is considered that the sequence of the MS2 viral capsid protein compared to that of the GA viral capsid protein is 59%. Also included is a VLP comprising a capsid protein having at least 86% sequence identity to SEQ ID NO: 1, when it is considered that the sequence of the MS2 viral capsid protein compared to that of the FR capsid protein is 86%. VLPs according to the present disclosure thus encompass those comprising a capsid protein having at least 15%, 16%, or 21% sequence identity with the amino acid sequence of wild type Enterobacteria phage MS2 capsid (SEQ ID NO: 1) based on a valid structure anchored alignment and is resistant to hydrolysis catalyzed by a peptide bond hydrolase category EC 3.4.

A VLP may thus comprise any of the MS2 capsid protein variants as described herein. Genetically modified capsid proteins consistent with those described herein can be produced for example by constructing at least one DNA plasmid encoding at least one capsid protein having at least one amino acid substitution, deletion or insertion relative to the amino acid sequence of the wild type MS2 capsid protein, making multiple copies of each plasmid, transforming a cell line with the plasmids; maintaining the cells for a time and under conditions sufficient for the transformed cells to express and assemble capsids encapsulating nucleic acids; lysing the cells to form a cell lysate; subjecting the cell lysate to hydrolysis using at least one peptide bond hydrolase, category EC 3.4; and removing intact capsids remaining in the cell lysate following hydrolysis to obtain capsids having increased resistance to at least one hydrolase relative to the wild type capsid protein. Following purification of the resulting, intact capsids, an amino acid sequence for each capsid protein may be determined according to methods known in the art.

The specialized capsids described herein can be used in research and development and in industrial manufacturing facilities to provide improved yields, since the purification processes used in both settings have the same matrix composition. Having such same composition mainly depends on using the same cell line in both research and development and manufacturing processes. However, differences in matrix composition due to using different cell lines are greatly reduced after proteolytic steps used in both research and development and manufacturing stages. This feature enables use of different cell lines in both stages with a minimal manufacturing yield penalty.

### EXAMPLES

The following non-limiting examples are included to illustrate various aspects of the present disclosure. It will be appreciated by those of skill in the art that the techniques disclosed in the following examples represent techniques discovered by the Applicants to function well in the practice of the invention, and thus can be considered to constitute preferred modes for its practice. However, those of skill in the art should, in light of the instant disclosure, appreciate that many changes can be made in the specific examples described, while still obtaining like or similar results, without departing from the scope of the invention. Thus, the examples are exemplary only and should not be construed to limit the invention in any way. To the extent necessary to enable and describe the instant invention, all references cited are herein incorporated by reference.

### Example A

### Capsid Coat Protein Variants

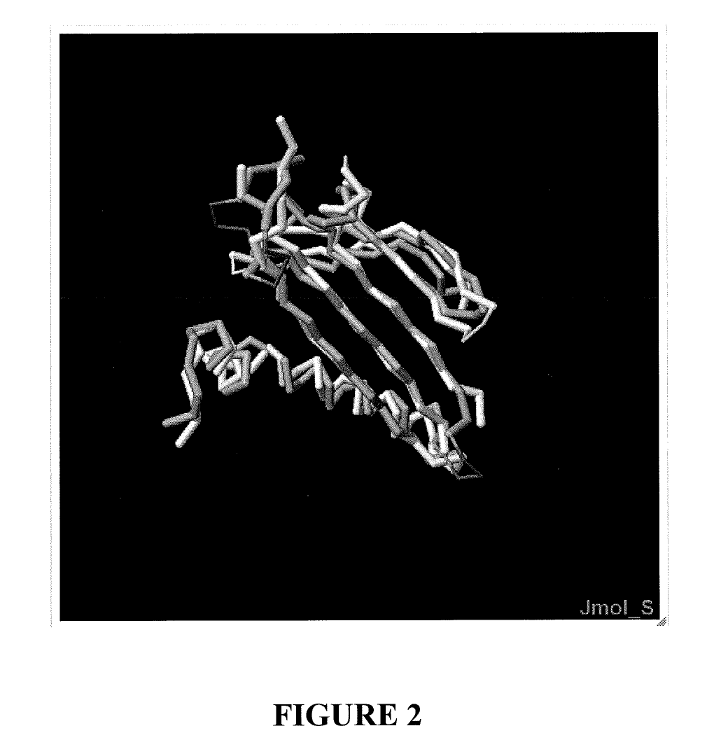

The MS2 viral capsid protein (SEQ. ID NO. 1) has a single folding domain and belongs to fold family d.85.1 (RNA bacteriophage capsid protein) of superfamily d.85 in the SCOP database, which includes leviviridae and alloleviviridae capsid proteins. Each capsid monomer in this family is made up of a 6-stranded beta sheet followed by the two helices (sometimes described as a long helix with a kink). 180 monomers assemble noncovalently to form an icosahedral (roughly spherical) viral capsid with a continuous beta-sheet layer facing the capsid interior and the alpha-helices on the capsid exterior. X-ray crystal structures have been solved and placed in the public domain for the enterobacteriophage MS2, GA (UniProt sequence identifier P07234) and FR (UniProt sequence identifier P03614) viral capsids and the capsid of MS2 formed from an MS2 dimer in which one C-terminus of one MS2 has been fused to the N-terminus of another, all d.85.1 family leviviridae coat proteins. The Protein Data Bank identifiers for these structures are 1AQ3 (SEQ ID NO: 2), 1GAV (SEQ ID NO: 3), 1FRS (SEQ ID NO: 4) and 2VTU (SEQ ID NO: 5), respectively, and alignment of these is shown FIG. 2. In this and all alignments described herein, the residue numbering is sequential residue numbering, for example SEQ ID NO. 1 starting with 0 for the lead Met (M) residue which is removed by the cell, as used for most PDB structures.

The sequences of MS2 viral capsid protein versus the GA and FR viral capsid proteins are 59% and 87% identical respectively. Only 56% of the sequence positions have identical sequence and topologically equivalent positions with respect to the backbone overlays when all three sequences are considered together. The rms deviation of the backbone conformations of MS2 viral capsid protein vs the GA and FR viral capsid monomers are under 1 A. The backbone rms deviation of 1AQ3 monomer A versus 1GAV monomer 0 is 0.89 Angstroms. The backbone fins deviation of 1AQ3 monomer A versus 1FRS monomer 30 A is 0.37 Angstroms. Comparisons were made using the freeware utility jFATCAT rigid (Prlic, et al, BioinfoHnatics 26,2983-2985 (2010); www.rcsb.org/pdb/workbench/workbench.do; www.rcsb.org/pdb/workbench/workbench.do), a tool familiar to practitioners of structure study protein available at the RCSB Protein Data Bank site in their standard workbench of protein structure tools. The overall fold of these proteins is identical. There are no insertions or deletions. Each protein in the crystallographic asymmetric unit is independently refined. Different, compositionally identical proteins within an asymmetric unit generally backbone rms deviations of 1 Angstrom or greater although topologically equivalent Calpha atoms of the core tend to differ by less, about 0.45 Angstroms (Cyrus Chothia and Arthur M Lesk (1986) EMBO J 5, 823-826). For example, 1AQ3 monomer A and 1AQ3 monomer B have rms deviation of 1.72 A (jFATCAT rigid) primarily because of conformational differences in the Lys66-Trp82 flexible loop region.

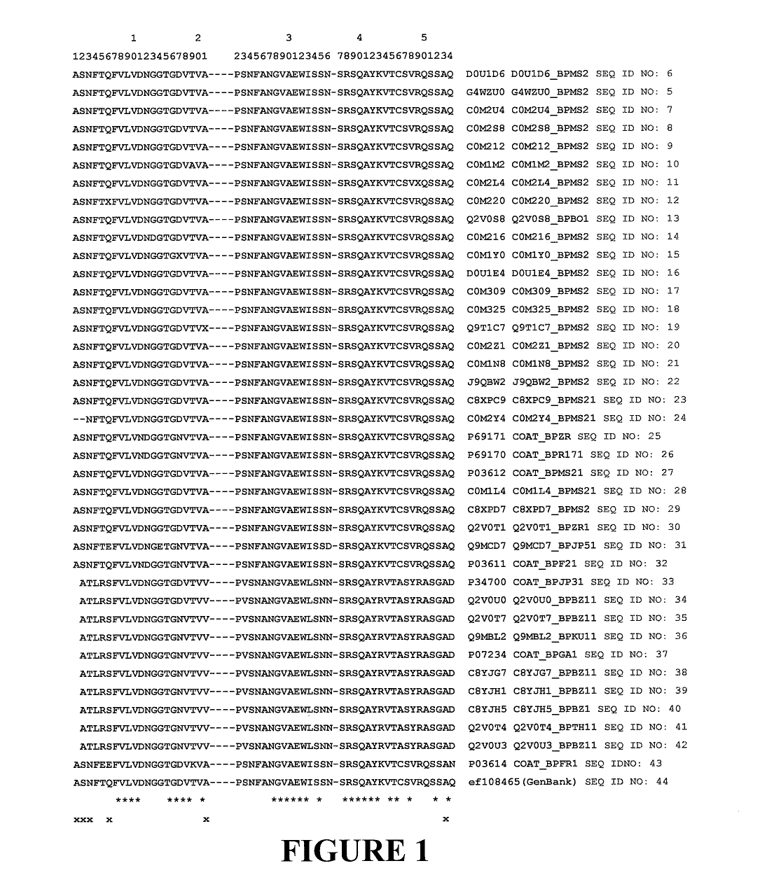

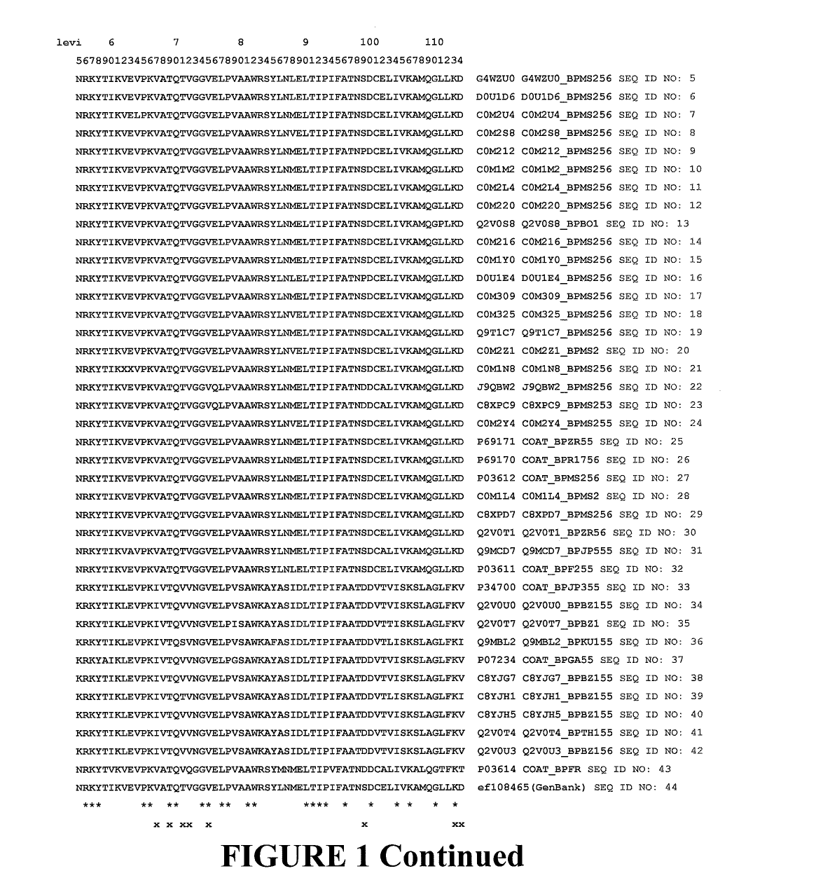

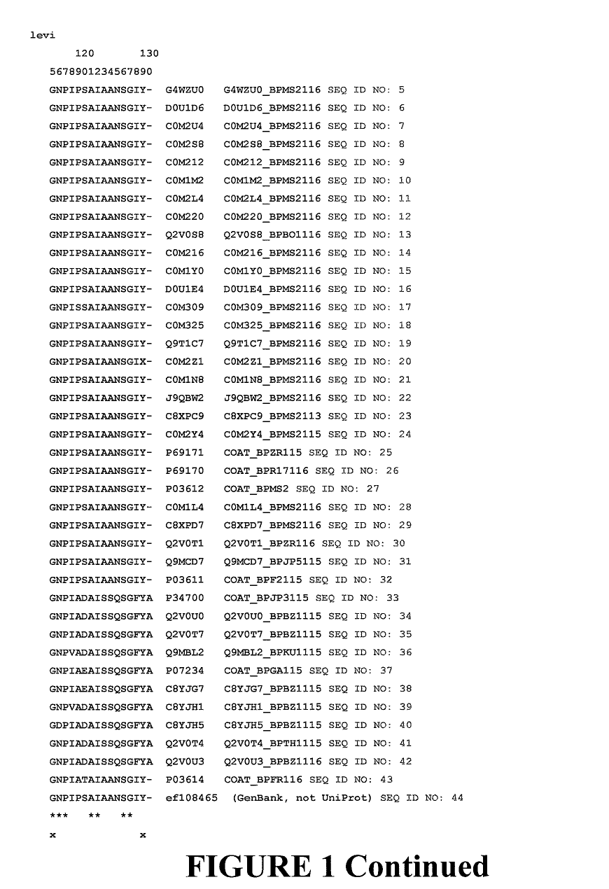

If sufficient members of a fold family have been identified, a clear picture of conserved residues, topologically equivalent residue positions within the sequences which seldom or never mutate within the family, emerges. Nonconserved positions can be expected to mutate from one sequence to another without disturbing the family fold, perhaps in conjunction with the concerted mutation of spatial neighbor(s) in the fold particularly if the sidechain packs against the sidechain(s) of the spatial neighbors. Conserved residues can be critical for fold stability, function or processing of the protein, for example proteolytic digestion. Some can be coincidentally conserved. GenBank (Dennis A. Benson, Ilene Karsch-Mizrachi, David J. Lipman, James Ostell, and David L. Wheeler (2005) Nucleic Acids Res 33, D34-D38) currently holds 353 leviviridae coat protein sequences. The alignment table shown in FIG. 1 shows the multiple alignment of 40 complete leviviridae coat protein sequences retrieved from the global protein sequence database UniProt (Universal Protein Resource, (The UniProt Consortium, Reorganizing the protein space at the Universal Protein Resource (UniProt) Nucleic Acids Res. 40: D71-D75 (2012)), http://www.uniprot.org) (See Table 1 below) and aligned with BLAST (threshold=10, Auto weighting array selection, no filtering, gaps allowed). All sequences except ef108465 were taken from UniProt. ef108465 came from GenBank (www.ncbi.nlm.nih.gov/genbank). In the alignment table, bottom of FIG. 1, an asterisk (*) indicates conserved residues, x is calculated to be substitutable based on sidechain solvent accessibility, hydrogen bonding requirements and backbone conformational constraints. Fifty-seven (57) residues in the sequences of these family members are conserved, or 45% of the sequences are identical to one another. Some of these sequences have an additional residue following the C-terminal Tyr129 residue of SEQ ID NO: 1, others have 1-2 residues removed from the N-terminus with respect to SEQ ID NO: 1. As is clearly shown in FIG. 1 there are no insertions or deletions within the fold.

TABLE 1
List of 41 complete leviviridae coat protein sequences from the UniProt database.

| Accession | Entry Name | Organism | SEQ ID NO: |
|---|---|---|---|
| G4WZU0 | G4WZU0_BPMS2116 | enterobacteria phage ms2 | SEQ ID NO: 5 |
| D0U1D6 | D0U1D6_BPMS2116 | enterobacteria phage ms2 | SEQ ID NO: 6 |
| C0M2U4 | C0M2U4_BPMS2116 | enterobacteria phage ms2 | SEQ ID NO: 7 |
| C0M2S8 | C0M2S8_BPMS2116 | enterobacteria phage ms2 | SEQ ID NO: 8 |
| C0M212 | C0M212_BPMS2116 | enterobacteria phage ms2 | SEQ ID NO: 9 |
| C0M1M2 | C0M1M2_BPMS2116 | enterobacteria phage ms2 | SEQ ID NO: 10 |
| C0M2L4 | C0M2L4_BPMS2116 | enterobacteria phage ms2 | SEQ ID NO: 11 |
| C0M2L4 | C0M2L4_BPMS2116 | enterobacteria phage ms2 | SEQ ID NO: 12 |
| C0M220 | C0M220_BPMS2116 | enterobacteria phage ms2 | SEQ ID NO: 13 |
| Q2V0S8 | Q2V0S8_BPBO1116 | enterobacteria phage bo1 | SEQ ID NO: 14 |
| C0M216 | C0M216_BPMS2116 | enterobacteria phage ms2 | SEQ ID NO: 15 |
| C0M1Y0 | C0M216_BPMS2116 | enterobacteria phage ms2 | SEQ ID NO: 16 |
| D0U1E4 | C0M216_BPMS2116 | enterobacteria phage ms2 | SEQ ID NO: 17 |
| C0M309 | C0M216_BPMS2116 | enterobacteria phage ms2 | SEQ ID NO: 18 |
| C0M325 | C0M216_BPMS2116 | enterobacteria phage ms2 | SEQ ID NO: 19 |
| Q9T1C7 | Q9T1C7_BPMS2116 | enterobacteria phage ms2 | SEQ ID NO: 20 |
| C0M2Z1 | C0M216_BPMS2116 | enterobacteria phage ms2 | SEQ ID NO: 21 |
| C0M1N8 | C0M216_BPMS2116 | enterobacteria phage ms2 | SEQ ID NO: 22 |
| J9QBW2 | C0M216_BPMS2116 | enterobacteria phage ms2 | SEQ ID NO: 23 |
| C8XPC9 | C8XPC9_BPMS2113 | enterobacteria phage ms2 | SEQ ID NO: 24 |
| C0M2Y4 | C0M2Y4_BPMS2115 | enterobacteria phage ms2 | SEQ ID NO: 25 |
| P69171 | COAT_BPZR115 | enterobacteria phage zr | SEQ ID NO: 26 |
| P69170 | COAT_BPR17116 | enterobacteria phage r17 | SEQ ID NO: 27 |
| P03612 | COAT_BPMS2 | enterobacteria phage ms2 | SEQ ID NO: 28 |
| C0M1L4 | C0M1L4_BPMS2116 | enterobacteria phage ms2 | SEQ ID NO: 29 |
| C8XPD7 | C8XPD7_BPMS2116 | enterobacteria phage ms2 | SEQ ID NO: 30 |
| Q2V0T1 | Q2V0T1_BPZR116 | enterobacteria phage zr | SEQ ID NO: 31 |
| Q9MCD7 | Q9MCD7_BPJP5115 | enterobacteria phage jp501 | SEQ ID NO: 32 |
| P03611 | COAT_BPF2115 | enterobacteria phage f2 | SEQ ID NO: 33 |
| P34700 | COAT_BPJP3115 | enterobacteria phage jp34 | SEQ ID NO: 34 |
| Q2V0U0 | Q2V0U0_BPBZ1115 | enterobacteria phage jp500 | SEQ ID NO: 35 |
| Q2V0T7 | Q2V0T7_BPBZ1 | enterobacteria phage sd | SEQ ID NO: 36 |
| Q9MBL2 | Q9MBL2_BPKU1115 | enterobacteria phage ku1 | SEQ ID NO: 37 |
| P07234 | COAT_BPGA115 | enterobacteria phage ga | SEQ ID NO: 38 |
| C8YJG7 | C8YJG7_BPBZ1115 | enterobacteria phage bz13 | SEQ ID NO: 39 |
| C8YJH1 | C8YJH1_BPBZ1115 | enterobacteria phage bz13 | SEQ ID NO: 40 |
| C8YJH5 | C8YJH5_BPBZ1115 | enterobacteria phage bz13 | SEQ ID NO: 41 |
| Q2V0T4 | Q2V0T4_BPTH1115 | enterobacteria phage th1 | SEQ ID NO: 42 |
| Q2V0U3 | Q2V0U3_BPBZ1116 | enterobacteria phage tl2 | SEQ ID NO: 43 |
| P03614 | COAT_BPFR116 | enterobacteria phage fr | SEQ ID NO: 44 |
| ef108465 | | enterobacteria phage r17 | SEQ ID NO: 45 |

Further, amino acid residues are distinguished by the identity of their sidechains. They share a common backbone and a common set of allowed backbone conformations (Kleywegt and Jones, Structure 4 1395-1400 (1996)), with two exceptions. Glycines can stably fold into backbone conformations disallowed to other amino acids because its sidechain consists of a single hydrogen atom. The proline sidechain is cyclized into a stiff ring which is covalently bound to its backbone nitrogen through elimination of its amide hydrogen, constraining proline to a small subset of backbone conformations with respect to the other amino acids and eliminating its ability to be a hydrogen bond donor.

The domain fold and domain association for assembly into capsids (for example of the amino sequence of SEQ ID NO: 1 is stabilized by the backbone hydrogen bonding patterns that define its secondary structural units, hydrogen bonds between sidechain and backbone atoms that stabilize local structure or bind neighboring secondary structure units (e.g. helices, strands, coil, loops, turns and flexible termini) together, hydrogen bonds between the atoms of different sidechains that stabilize local structure or bind neighboring secondary structure units (e.g. helices, strands, coil, loops, turns and flexible termini) together and the close packing of hydrophobic sidechain atoms that serves to both energetically stabilize the fold through van der Waals interactions and to prevent solvent penetration into the fold which might lead to destabilization and local unfolding. The sidechains of the remaining residues do not participate in domain fold maintenance or in domain-domain interactions. So long as their backbone conformations do not have special requirements satisfied only by Gly or cis-Pro in order to participate in the domain fold, these residues can be mutated, singly or as a group, without substantially affecting the final domain fold or the overall topology of its surface, and can be identified as a class unequivocally by surface accessibility calculations performed on known structures (See, e.g., Summers, Carlson, and Karplus, JMB 196: 175-198 (1987); Fraczkiewicz and Braun, J Comp Chem 19, 319 (1998)), followed by hydrogen bond analysis of known structures, all conventional techniques in the study of protein structure and function.

Using two MS2 capsid structures from the Protein Data Bank for examination, 1AQ3 (SEQ ID NO: 2) of an icosahedral capsid containing RNA and 2VTU (SEQ ID NO: 5) of a stable octahedral capsid formed by 2 MS2 capsid protein monomers fused C-terminus to N-terminus to form the single chain protein 2 domain protein MS2-(ΔS2)MS2, 17 residues (Ala1, Ser2, Thr5, Gln6, Ala21, Ala53, Val67, Thr69, Thr71, Val72, Val75, Ser99, Glu102, Lys113, Asp114, Gly115, Tyr129) were identified which have highly solvated sidechain positions (Fraczkiewicz and Braun; server http://curie.utmb.edu/getarea with 1.4 Angstroms solvent probe, no gradient, 2 area/energy per residue); do not participate in hydrogen bonds with other parts of the capsid (hydrogen bonds calculated in the widely used freeware software visualization package Chimera (Eric F. Pettersen, Thomas D. Goddard, Conrad C. Huang, Gregory S. Couch, Daniel M. Greenblatt, Elaine C. Meng, Thomas E. Ferrin (2004) J Comp Chem 25, 1605-1612) with hydrogen bond criteria relaxed by 0.5 Angstroms and 30 deg); and with backbone conformations allowed by all amino acid residues except proline. When the subset of these 17 residues is compared to the structural alignment of the enterobacteria phage MS2, wherein GA and FR capsid sequences and residues which have mutated in the enterobacteria phage GA or FR capsid sequences are disregarded, leaving 6 positions remaining which are putatively susceptible to mutation without effecting the structure or function of the monomers or their ability to assemble into stable capsids. This represents 52% sequence identity to wild-type enterobacteria phage MS2 capsid protein (SEQ ID NO: 1).

The insertion and/or deletion of residues within secondary structure elements (helices, strands, turns with defining hydrogen bonding patterns and structured loops, e.g. omega loops) cause those elements to lose their defining hydrogen bonding or hydrophobic packing patterns or force a change in their hydrogen bonding or hydrophobic packing patterns which can alter stability, shape and/or function from the original protein sequence. This can disrupt packing and affect the global stability of a fold. On the other hand, unstructured loops, random coils and N- and C-termini which have surface exposure but do not provide critical stabilization to the rest of the protein fold (frequently via the packing of sidechains against structured elements or the shielding of interacting faces of adjacent structured elements from solvent or in the case of capsids, cargo) are excellent candidates for (1) residue deletion if significant repositioning of the joined structured elements is not required, (2) insertion of amino acid residues if the addition of residues will not significantly alter the relative disposition of structured elements in the fold or screen surface exposed residues from satisfying their hydrogen bonding capacity with hydrogen bond donors or acceptors in the protein's environment or (3) the incorporation of naturally-occurring amino acid mutation(s) or mutation(s) to nonnative residues which can be covalently linked to useful moieties, e.g. fluorophores, phosphorescent groups, polyethylene glycols, affinity tags and reporter groups. Of course, such insertions, deletions and mutations can occur within a single suitable element concurrently or in any combination and their incorporation may give rise to a protein with improved characteristics. One way to distinguish optimal spots for insertion and/or deletions is to scan the multiple alignments of closely related sequences for insertions and/or deletions. Aside from N- and C-terminal additions and deletions, the known leviviridae coat protein sequences do not have insertion or deletions with respect to each other. This does not mean insertion and/or deletions cannot occur. One simply must examine more distant members of the structure/function or fold family to identify likely positions for such insertions or deletions.

The simplest multiple alignment algorithms are usually available to the general public at the public domain sequence and structure data bases. These algorithms can correctly align sequences that share a very low percent identity if the sequence space is populated by a continuous spectrum of sequences from a high percent identity, for example 90%, to a low identity, for example 20%. These algorithms tend to fail to correctly align clusters of sequences with the same fold when those cluster share a low percent identity; however, such clusters can be successfully and unequivocally aligned if the x-ray crystal structure of one or more members of each cluster has been solved and well refined. By optimally superimposing backbone atoms of the secondary structure elements of the structures of proteins closely related by fold but distantly related by sequence, a one-to-one correspondence between their sequences is clearly defined and the high percent identity clusters successfully generated by sequence alignment protocols can be anchored to the pairwise alignment resulting from the backbone superposition and a correct global sequence alignment for the fold family generated resulting in a topologically meaningful alignment of the fold family members (Arthur M Lesk, Michael Levitt, Cyrus Chothia (1986), Prot Eng 1, 77-78). By examining the global sequence alignment, a comprehensive picture of where the fold will tolerate insertion and/or deletion without compromising its form or function can be viewed.

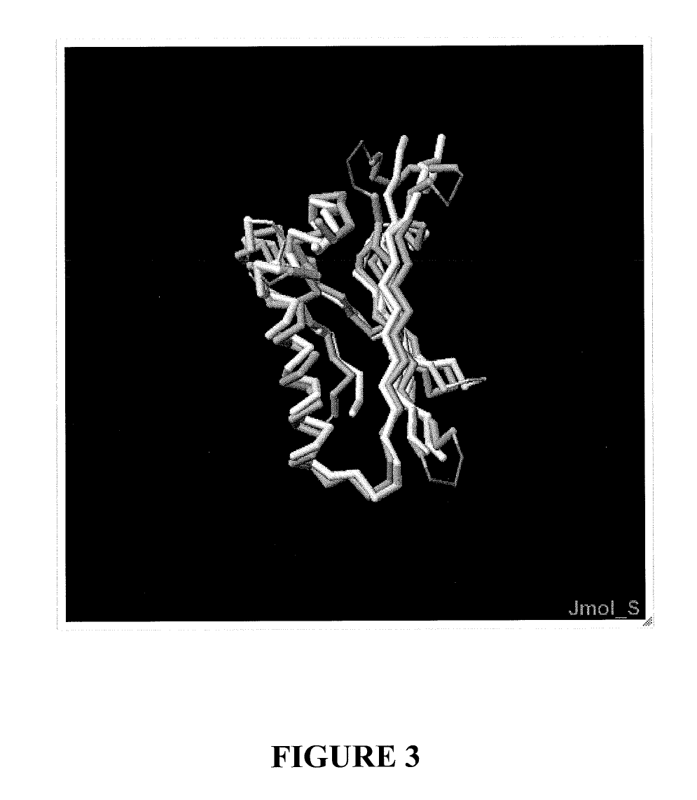

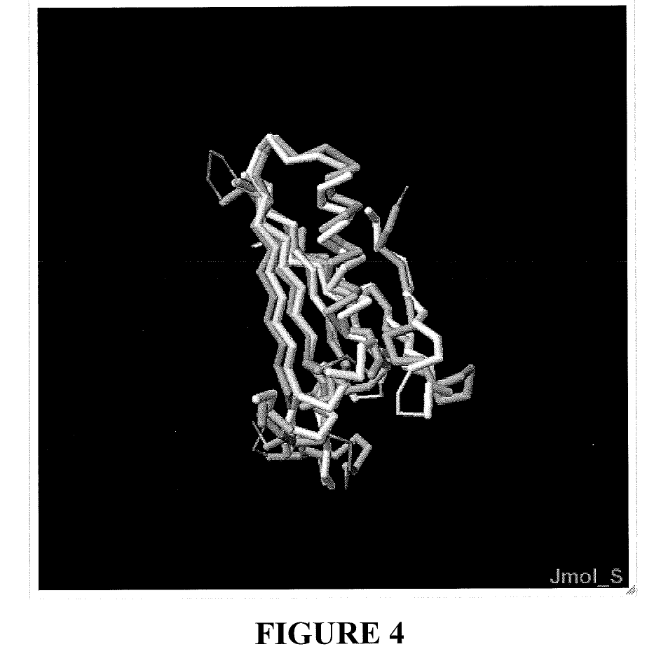

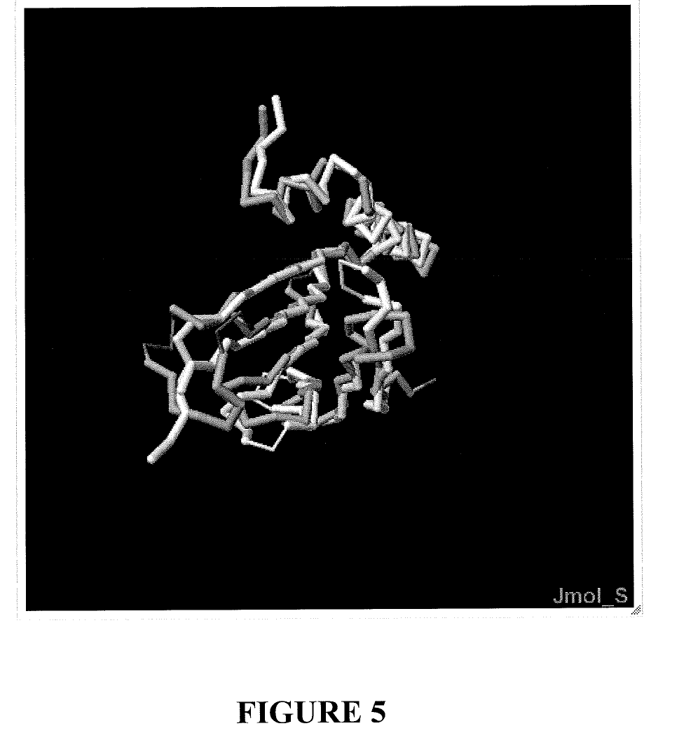

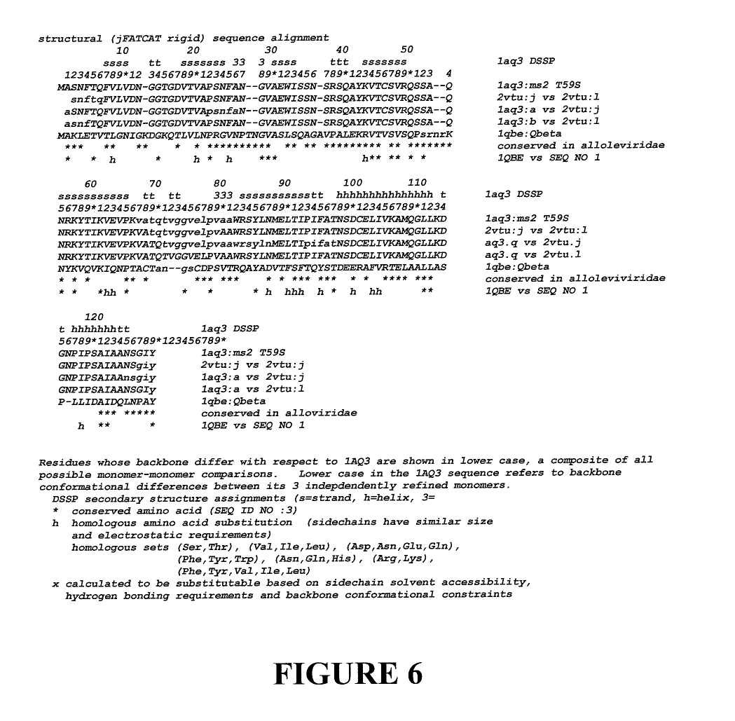

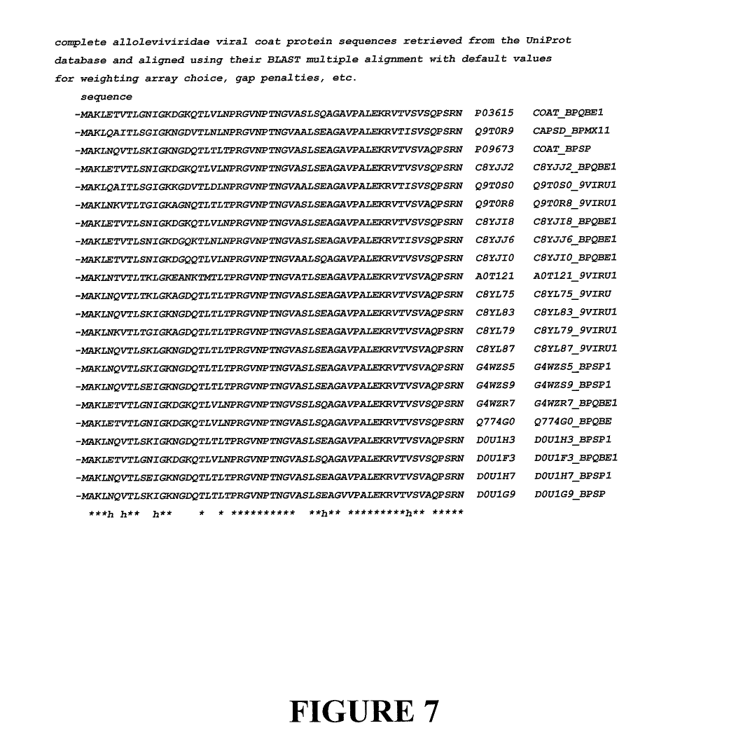

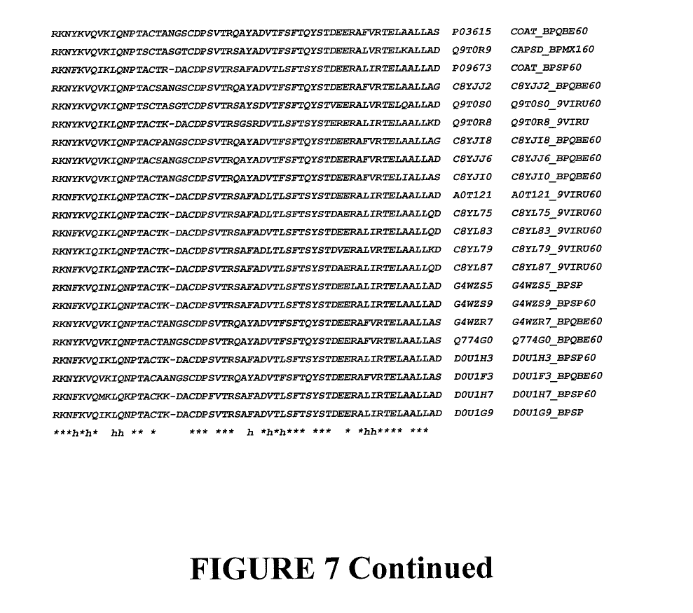

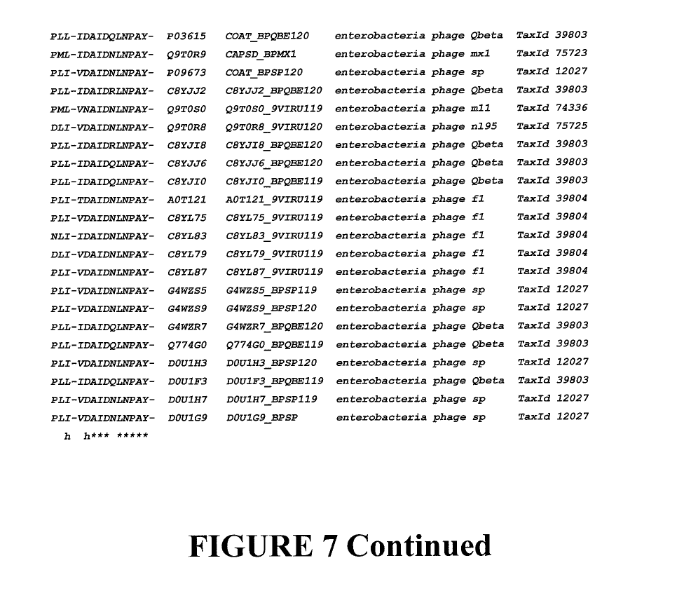

The alloleviviridae coat proteins belong to the same fold family as the leviviridae coat proteins (fold family d.85.1) and also assemble into icosahedral capsids comprised of 180 monomers. The multiple alignments of the sequences of alloleviviridae coat proteins deposited in UniProt are shown in the alignment table in FIG. 7. Sixty percent (60%) of the alloleviviridae coat protein sequence is conserved. The coat proteins of levi- and alloleviviridae are both about 130 amino acid residues long but because the percent of identical residues is low, about 20%, multiple sequence alignment algorithms typically fail to correctly align the allolevi-against the leviviridae sequences. A simple way to recognize this is to reverse the sequences and then use the same protocol to align the reversed sequences. The multiple alignments of the sequences and reversed sequences will not agree. This difficulty can be circumvented by examining representative structures. An x-ray crystal structure of a capsid of alloleviviridae Qbeta (PDB-ID:1QBE) (SEQ ID NO: 46, see below) has been deposited in the public domain database, RCSB Protein Data Bank (http://www.rcsb.org). The independently refined monomers of 1QBE were fit to the independently refined monomers of 1AQ3 by minimizing the rms deviation between Calpha atoms using the jFATCAT comparison tool at the RCSB Protein Data Bank. The rms deviation is in the range 2.33-2.76 Angstroms depending upon which of the independently refined monomers is compared, primarily due to differences in the backbone disposition of N-terminal residues 1-3 and segments 8-18, 26-28, 50-55 and 67-76 (numbering references the topologically equivalent residues in the MS2 structure 1AQ3) which connect secondary structure elements, as shown in FIGS. 3-6 and described in the accompanying figure descriptions. The backbone rms deviation measured by jFATCAT for independently refined monomers in 1AQ3 is 1.72 Angstroms due to conformational differences in the same regions. The topological alignment is shown in the table, secondary structure assignment by hydrogen bonding pattern (DSSP, W Wolfgang Kabsch and Christian Sander (1983), Biopolymers 22, 2577-2636) is indicated for 1AQ3 and segments that show the greatest deviation either because the refined backbone conformations are substantially different or because the segments were too mobile to be localized in electron density during refinement are provided in lower case. Regions which show backbone flexibility in the crystal environment are also excellent candidates for insertion/and or deletion because if the interactions between these residues and the rest of the fold was important for fold stabilization, their electron density would be localized. Appending the same information for 2VTU provides further insight into segments best adapted to accommodate change. These comparisons are captured symbolically in FIG. 7 which shows alignment of 1AQ3 versus 2VTU versus 1QBE.

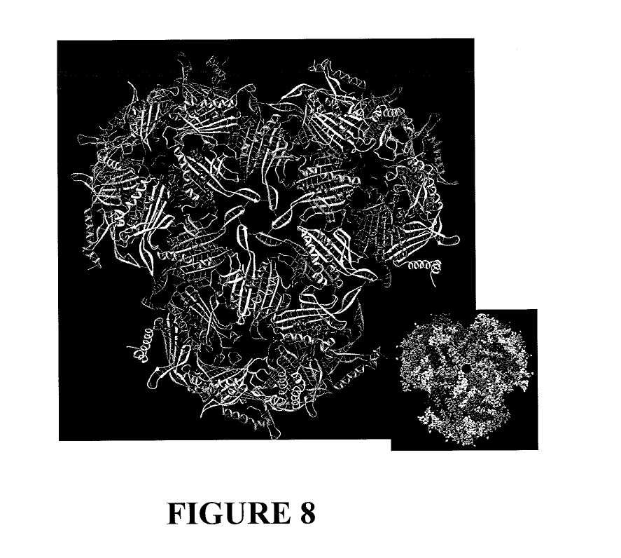

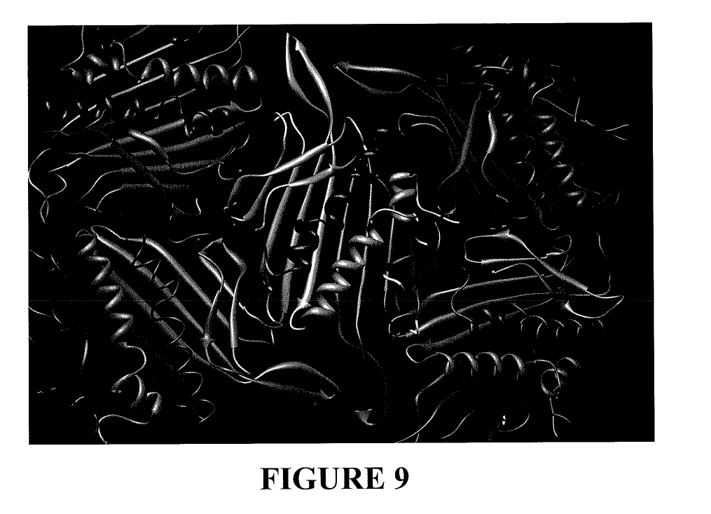

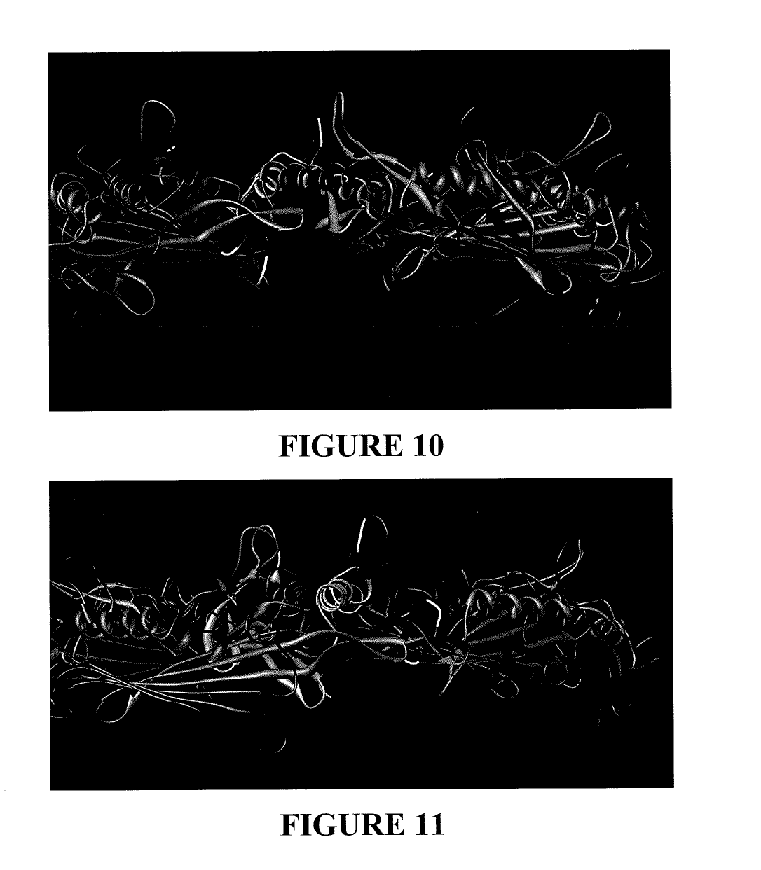

Examination of the 1AQ3 and 1QBE monomers provides the following insights, as further illustrated by reference to FIGS. 8-11 and their respective descriptions. All residue numbers are given with respect to the monomers in 1AQ3.

This also means that the fold of SEQ ID NO: 1 Enterobacteria phage MS2 coat protein is preserved down to 21% identity versus the sequence of 1 QBE Enterobacteria phage coat protein Qbeta (SEQ ID NO: 46) and 16% identity with respect to the conserved residues for all of the alloleviviridae coat protein sequences referenced here. Only one of the highly solvated sidechain positions calculated earlier, sidechains which do not participate in hydrogen bonds with other parts of the capsid and whose backbone conformations are allowed by all amino acid residues except proline, Y129 (in SEQ ID NO: 1 numbering) remains conserved. Its backbone position and sidechain packing is substantially changed in the octahedral Enterobacteria phage MS2 capsid structure formed by the fused MS2 dimer (2VTU). After this change is considered, the threshold amino acid sequence percent identity is lowered to 15%. See the alignment tables in FIG. 2 and FIG. 7 (1AQ3 versus 2VTU versus 1QBE, and allolevi multiple sequence alignment tables for clarification). All percent similarities in this paragraph are valid only in the context of structure anchored alignments.

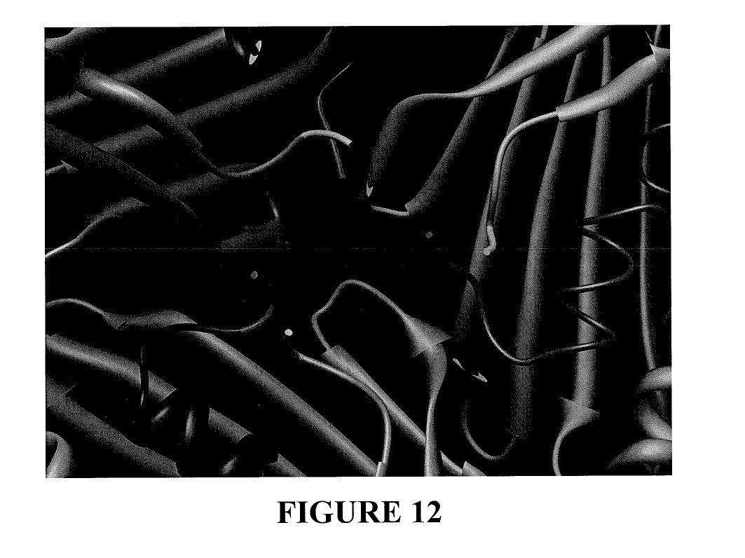

N-terminal residues 1-3 can satisfy their hydrogen bonding potential with the C-terminal residue 129 and water and vice versa; therefore, it should be possible to delete some or all of these residues and form stable VLPs with the truncated proteins. FIG. 12 shows backbone ribbon diagrams of 3 noncovalent Enterobacteria phage MS2 noncovalent dimers packed around a symmetry point in the assembled icosahedral capsid (dimer one right, light and dark chains; dimer two bottom, dark and medium chains; dimer three upper right, light gray and dark chains). All chain N-termini are shaded dark, all C-termini are shaded light. The proximity of the termini mean that that the sequences of the monomers can be fused into a single chain to form a covalent dimer, either as done for 2VTU by appending one monomer after the other, i.e., creating a single protein chain that consists of (monomer residues 1-129-monomer residues 1-129) or by adding additional linking residues between the monomer sequences (monomer 1-129-linker residues-monomer 1-129) as long as the relative chain directions (from N- to C-terminus) allow a continuous peptide chain to be formed from the concatenated monomers. A monomer-monomer concatenation without the addition of linker residues was solved (PDB-ID:2VTU). In 2VTU each noncovalent dimer has been engineered into a single protein; however, since the Calpha's of residues 2 and 129 are around 6 Angstroms apart, barely close enough to join with a linking segment without disturbing the fold (the Calpha-Calpha distance is constrained to about 3.8 Angstroms because of the resonance forms of the peptide unit) and in some monomers their backbones hydrogen bond with each other. The beta-sheet side of each dimer (covalent or noncovalent) forms the interior wall of the capsid. The geometry of a beta sheet can be defined by the curvature of the sheet (Cyrus Chothia, Jiri Novotny, Robert Bruccoleri, Martin Karplus (1985) J Mol Biol 186, 651-663). The tight coupling in 2VTU (MS2-(ΔS2)MS2) constrains the beta sheet to a lower curvature giving rise to an octahedral rather than an icosahedral capsid. The incorporation of a linker between monomers of 0-6 residues would provide enough flexibility to allow the covalent dimer to relax into the conformation required for an icosahedral capsid, with physical properties likely to be more closely related to the icosahedral noncovalent capsid structure. Generally, the linker will be 1-6 residues, however, for example, the covalent dimer of 2VTU actually has Ser2 deleted in the second copy. To restore icosahedral capsid geometry under identical crystallization conditions would a linker of at least 1 residue. In such cases the linker length would be 1-6 residues.

Residues chosen for the linker should have small sidechains to avoid steric strain which can be caused by a large number of atoms packing into a relatively small volume. Strain can also be minimized by avoiding the choice of amino acid residues with smaller backbone conformational space, for example proline. Avoiding strain can translate into a protein which folds more quickly or more efficiently. Bulkier and charged sidechains, particularly in the middle section of longer loops tend to be binding targets for proteases. Gly-containing linkers are preferred.

From FIG. 12 it is also clear that the C-terminus of one monomer can be linked to the N-terminus of a monomer participating in the neighboring noncovalent dimer and a stable icosahedral capsid could still form as long as the linker was of appropriate length and flexibility and did not contain a potential cleavage site accessible by proteases in the capsid environment. In fact, three monomers could be linked with appropriate linkers and still form this section of capsid, because the light gray, and dark gray monomers of FIG. 12, are also the asymmetric unit of the capsid. Three monomers concatenated end to end with appropriate linking segments should also be able to form a stable icosahedral capsid.

N-terminal residues 1-3 can satisfy their hydrogen bonding potential with the C-terminal residue 129 and water and vice versa; therefore, it should be possible to delete some or all of these residues and form stable VLPs with the truncated proteins or alternatively with the corresponding potential linker lengths extended by the number of deletions in concatenated proteins.

Accordingly, the present disclosure encompasses VLPs comprising a capsid comprising a capsid protein which is a variant of wild type Enterobacteria phage MS2 capsid (SEQ ID NO: 1) and is resistant to hydrolysis catalyzed by a peptide bond hydrolase category EC 3.4. For example, a VLP may comprise a capsid protein with the amino acid sequence of wild type Enterobacteria phage MS2 capsid (SEQ ID NO: 1) except that the A residue at position 1 is deleted. A VLP may comprise a capsid protein with the amino acid sequence of wild type Enterobacteria phage MS2 capsid (SEQ ID NO: 1) except that the A residue at position 1 is deleted and the S residue at position 2 is deleted. A VLP may comprise a capsid protein with the amino acid sequence of wild type Enterobacteria phage MS2 capsid (SEQ ID NO: 1) except that that the A residue at position 1 is deleted, the S residue at position 2 is deleted and the N residue at position 3 is deleted. A VLP may comprise a capsid protein with the amino acid sequence of wild type Enterobacteria phage MS2 capsid (SEQ ID NO: 1) except that the Y reside at position 129 is deleted. A VLP may comprise a capsid protein with the amino acid sequence of wild type Enterobacteria phage MS2 capsid (SEQ ID NO: 1) but having a single (1) amino acid deletion in the 112-117 segment. A VLP may comprise a capsid protein with the amino acid sequence of wild type Enterobacteria phage MS2 capsid (SEQ ID NO: 1) but having a single (1) amino acid deletion in the 112-117 segment. A VLP may comprise a capsid protein with the amino acid sequence of wild type Enterobacteria phage MS2 capsid (SEQ ID NO: 1) but having a 1-2 residue insertion in the 65-83 segment and is resistant to hydrolysis catalyzed by a peptide bond hydrolase category EC 3.4. A VLP may comprise a capsid protein with the amino acid sequence of wild type Enterobacteria phage MS2 capsid (SEQ ID NO: 1) but having a 1-2 residue insertion in the 44-55 segment. A VLP may comprise a capsid protein with the amino acid sequence of wild type Enterobacteria phage MS2 capsid (SEQ ID NO: 1) but having a single (1) residue insertion in the 33-43 segment and is resistant to hydrolysis catalyzed by a peptide bond hydrolase category EC 3.4. A VLP may comprise a capsid protein with the amino acid sequence of wild type Enterobacteria phage MS2 capsid (SEQ ID NO: 1) but having a 1-2 residue insertion in the 24-30 segment. A VLP may comprise a capsid protein with the amino acid sequence of wild type Enterobacteria phage MS2 capsid (SEQ ID NO: 1) but having a single (1) residue insertion in the 10-18 segment. A VLP may comprise a capsid protein monomer sequence concatenated with a second capsid monomer sequence which assembles into a capsid which is resistant to hydrolysis catalyzed by a peptide bond hydrolase category EC 3.4. A VLP may comprise a capsid protein monomer sequence whose C-terminus is extended with a 0-6 residue linker segment whose C-terminus is concatenated with a second capsid monomer sequence, all of which assembles into a capsid which resistant to hydrolysis catalyzed by a peptide bond hydrolase category EC 3.4. Suitable linker sequences include but are not limited to -(Gly)x-, where x is 0-6, or a Gly-Ser linker such as but not limited to -Gly-Gly-Ser-Gly-Gly-, -Gly-Gly-Ser and -Gly-Ser-Gly-. A VLP may further comprise a capsid protein monomer sequence concatenated with a third capsid monomer sequence which assembles into a capsid which is resistant to hydrolysis catalyzed by a peptide bond hydrolase category EC 3.4. Again, in the capsid protein, the C-terminus can be extended with a 0-6 residue linker segment whose C-terminus is concatenated with a third capsid monomer sequence, all of which assembles into a capsid which is resistant to hydrolysis catalyzed by a peptide bond hydrolase category EC 3.4. One or both linker sequences can be selected from -(Gly)x-, where x=0-6, or a Gly-Ser linker selected from -Gly-Gly-Ser-Gly-Gly-, -Gly-Gly-Ser and -Gly-Ser-Gly-. For example, in one or both linker sequences, the linker is -(Gly)x-, and x is 1, 2 or 3. A VLP may comprise one or more coat protein sequences which are N-terminally truncated by 1-3 residues, wherein a linker sequence is lengthened by the number of residues deleted from the N-terminus of the following protein, wherein the linker sequence is -(Gly)x-, wherein x=0-6. For example, a VLP may comprise one or more coat protein sequences which is C-terminally truncated by 1 residue and then a linker sequence is lengthened by the 1 residue, wherein the linker sequence immediately following is -(Gly)x-, wherein x=0-6. A VLP may comprise two coat protein sequences, wherein the first coat protein sequence in a concatenated dimer is C-terminally truncated by 1 residue and a linker sequence is lengthened by the one residue or wherein the first and/or second coat protein sequence in the concatenated trimer is C-terminally truncated by 1 residues, wherein the linker sequence is -(Gly)x-, wherein x=0-6.

### Example B

### Controlling Proteolytic Loss of VLPs by Hydrolases

Additional examples of viruses with capsids proteins of special interest for forming the VLPs include:
Satellite tobacco necrosis virus (Satellivirus)(Lane, S. et al. (2011) J. Mol. Biol. Construction and Crystal Structure of Recombinant STNV Capsids, 413: 41-50; Ford, R. et al. (2013) J. Mol. Biol.Sequence-Specific, RNA-Protein Interactions Overcome Electrostatic Barriers Preventing Assembly of Satellite Tobacco Necrosis Virus Coat Protein 425: 1050-1064);
Physalis mottle virus (Tymovirus) (Sastry, M. et al. (1997) J. Mol. Biol. Assembly of Physalis Mottle Virus Capsid Protein in Escherichia coli and the Role of Amino and Carboxy Termini in the Formation of the Icosahedral Particles, 272: 541-552);
Maize rayado (Marafivirus) virus (Hammond R. and Hammond J. (2010) Maize rayado (Marafivirus) fino virus capsid proteins assemble into virus-like particles in Escherichia coli, Virus Research 147: 208-215); and
Macrobrachium rosenbergii nodavirus (Alphanodavirus) (Goh, Z. et al. (2011) Journal of Virological Methods, Virus-like particles of Macrobrachium rosenbergii nodavirus produced in bacteria, 175: 74-79; Zhong, W. et al. (1992) Proc. Natl. Acad. Sci. USA, Evidence that the packaging signal for nodaviral RNA2 is a bulged stem-loop, 89: 11146-11150).

I. Enzymes

The EC 3.4 hydrolases catalyze breakage of the protein backbone peptide bond. Binding the substrate in a highly constrained conformation at the position of backbone cleavage is a necessary first step. Enzymes have evolved in two ways to accomplish this quickly and efficiently. First, active sites have evolved to be somewhat sequestered clefts or deep depressions on the enzyme surface so that solvent not participating in the catalytic event can be excluded from the site of chemistry. Second, many hydrolases selectively bind several residues near the substrate cleavage site to increase efficiency by reducing local entropy and lowering the reaction barrier for cleavage. Hydrolases can often be distinguished by their binding preferences; some are exquisitely specific, breaking only a single bond in a single protein, while others cleave broadly.

The most broadly specific hydrolases can digest a protein into many fragments. As digestion progresses, the increasing number of cleavages can lead to local unfolding which exposes more potential cleavage sites to the hydrolase and accelerates the digestion process to conclusion. However, when cleavage is limited the target protein can retain its fold and function even though it has sustained backbone breakages. For example, specific hydrolysis liberates active proteins from their proforms and enzymatic deglycosylation can introduce accidental backbone cleavage, often near leucines, without detrimentally affecting the protein fold or function. In x-ray structures solved for deglycosylated proteins, these cleavage sites are seen as missing density. Age of a protein can be estimated by measuring the degree of protein deamidation including isoaspartate formation, which involves backbone cleavage.

In the expression Xaa′-XaalYaa, the symbol “|” denotes the hydrolase cleavage site. Xaa residues are preferred immediately before the cleavage site in the chain, Yaa residues are preferred immediately after the cleavage site and Xaa′ precedes Xaa in the chain. Some hydrolases have preferred residues at this site as well. Known cleavage preferences are cataloged in the Integrated relational Enzyme database (IntEnz), http://www.ebi.ac.uk/intenz/or are available from the International Union of Biochemists and Molecular Biologists official Enzyme Nomenclature publication http://www.chem.qmul.ac.uldiubmb/enzyme/index.html. Alternatively enzyme cleavage preferences could be taken from a different database of enzyme cleavage preferences, manufacturer's product sheets or from cleavage prediction software, for example PeptideCutter http://web.expasy.org/peptide_cutter, Gasteiger E, Hoogland C, Gattiker A, Duvaud S, Wilkins M R, Appel R D, Bairodch A; Protein Identification and Analysis Tools on the ExPASy Server; J M Walker (ed): The Proteomics Protocols Handbook, Humana Press (2005)). Software like PeptieCutter assumes a denatured form as an initial condition.

TABLE 2
Cleavage preferences of some common industrial proteases.

| Enzyme | Xaa′ | Xaa | Yaa |
|---|---|---|---|
| peptidase K | | large uncharged side | no preference |
| | | chains | |
| streptogrisin | | Tyr, Trp, Phe, Leu | no preference |
| A | | | |
| streptogrisin | | Arg, Lys | no preference |
| B | | | |
| pepsin A | | aromatic, | aromatic, |
| | | hydrophobic side | hydrophobic side |
| | | chains | chains |
| papain | large hydrophobic | no preference | no preference |
| | side chains | | |
| subtilisin A | | large uncharged side | no preference |
| | | chains | |

Presence of preferred residues in the protein is a necessary but insufficient condition for cleavage. Aside from the proteolytic site, hydrolases tend to have spheroid, prolate spheroid or oblate spheroid shapes of intermediate size whose interior is tightly packed with the atoms of the hydrolase. A proteolytic event also requires the enzyme to be able to approach and bind the substrate, i.e., the location of the cleavage site on the surface of the target protein must be able to accommodate the excluded volume of the hydrolase, here estimated as follows. A Cartesian coordinate set of a representative x-ray structure of the hydrolase solved at high resolution and of good quality is selected, preferably of the hydrolase in complex with a peptide, peptide analog or peptide mimetic bound in its active site and most preferably in complex with another protein bound in its active site. Using any protein visualization software of choice that can produce distance measurements, or by applying basic analytic geometry to the coordinate set, the hydrolase is centered at its catalytic residues, then oriented with the maximum area of entrance to the active site pointed down along the negative z-axis and with the protein, peptide, peptide analog or peptide mimetic backbone near the active site positioned horizontally (along the x-axis). If the hydrolase does not participate in a complex, the approximate position of a putative bound substrate or inhibitor backbone near the active site positioned horizontally can be chosen as an x-axis. The y-axis measures depth or width of the hydrolase and the z-axis the distance the targeted protein must penetrate the hydrolase binding cleft in order to bind at its active site. The footprint of the bound hydrolase on its targeted protein can then be conservatively estimated by measuring the maximum outer diameter of the hydrolase backbone along the x- and y-axes between the lowest, outermost hydrolase backbone atoms and the top (or back) of the binding pockets which accommodate substrate. The volume described in this way cannot be excluded by the volume of the target protein, in this case the formed viral capsid, if an enzymatic cleavage is to occur.

TABLE 3
Approximate footprint of some common industrial proteases.

| | | breadth | depth |
|---|---|---|---|
| Enzyme | PDB-ID | (\acute{Å}) | (\acute{Å}) |
| peptidase K | 2HPZ | ~30 | ~35 |
| streptogrisin A | 4SGA | ~25 | ~35 |
| streptogrisin B | 2QA9 | ~25 | ~32 |
| pepsin A | 1PSA | ~30 | ~55 |
| papain | 2CIO | ~30 | ~40 |
| subtilisin A | 1YU6 | ~30 | ~40 |

Finally, proteolysis becomes problematic in industrial applications when enzyme turnover numbers are high or the hydrolase is in prolonged contact with the product protein. Time of contact can be regulated by optimizing the manufacturing process. Turnover numbers in a given solvent medium are an inherent characteristic of the protein, so the only option that remains for controlling enzyme efficiency is by limiting the possible number of productive encounters a hydrolase can have with its target protein by eliminating the presence of binding motifs in the locations on the target protein surface which can be most readily bound productively by the hydrolase. These tend to be loops, exterior strands of beta sheet, helix caps or random coils on the protein surface which extend away from the protein surface into the solvent environment. Increased flexibility in these segments due, for example to backbone atoms hydrogen bonding with solvent components rather than other atoms of the protein, sidechain atoms hydrogen bonding primarily with solvent components, the presence of one or more glycine residues in the segment, the absence of bulky residues or the presence of multiple residues in close proximity with the same polarity as solvent, generally increases the probability of productive encounters.

II. VLPs

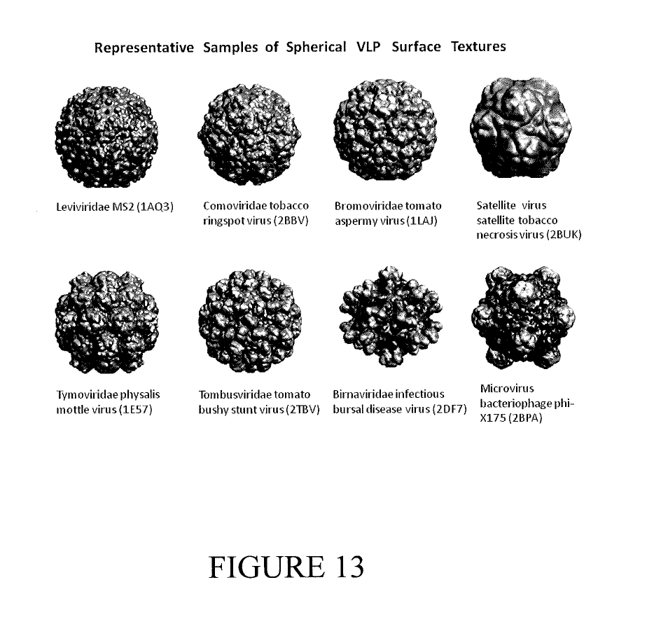

Structural studies involving various techniques (e.g., electron microscopy, crystallography, etc.) have shown viral capsids to be highly textured, but also susceptible to being qualitatively sorted into categories based on their gross surface shapes and texture patterns, coupled with the texture penetration depth with respect to an idealized capsid shape, such as, for example, a sphere (FIG. 13 shows sample textures of spherical capsids, images taken from ViperDB (Mauricio Carrillo-Tripp, Craig M. Shepherd, Ian A. Borelli, Sangita Venkataraman, Gabriel Lander, Padmaja Natarajan, John E. Johnson, Charles L. Brooks III and Vijay S. Reddy (2009) VIPERdb2: an enhanced and web API enabled relational database for structural virology. Nucleic Acid Research 37, D436-D442; http://viperdb.scripps.edu).

Thus, aside from the more commonly used gene structure approach, it is also possible to categorize viral capsids by the fold classification of the folding domains of the individual capsid proteins. An extensive public domain database of protein domain folds, the Structural Classification of Proteins (SCOP) database (Alexey G Murzin, Curr Opin Struct Biol (1996) 6, 386-394) of solved protein structures in the public domain is maintained online at http://scop.berkeley.edu and regularly expanded as new solved structures enter the public domain (Protein Data Bank [F. C. Bernstein, T. F. Koetzle, G. J. Williams, E. E. Meyer Jr., M. D. Brice, J. R. Rodgers, O. Kennard, T. Shimanouchi, M. Tasumi, “The Protein Data Bank: A Computer-based Archival File For Macromolecular Structures,” J. of Mol. Biol., 112 (1977): 535], http://www.rcsb.org). Importantly, a domain fold class is not restricted to particular structure/function families of proteins or gene structure. It is a basic building block in the formation of the characteristic three-dimensional, biologically active shape of folded amino acid sequences. Fold classifications for known viral capsids as reported by SCOP are shown in the Table below of SCOP viruses, where the terms used as fold descriptions are familiar to knowledgeable practitioners.

TABLE 4
SCOP Viruses

| | | |
|---|---|---|
| | secondary structure | |
| SCOP structure class | elements | fold description |

ALL-ALPHA DOMAINS

| | | |
|---|---|---|
| poliovirus core protein 3a, | 4 helix | bundle with righthand twist; |
| soluble domain | | closed |
| hepatis B viral capsid | 5 helix | 4 helix bundle; array, helix- |
| | | turn-helix dimer |
| flavivirus capsid protein C | 5 helix | righthand superhelix; |
| | | swapped dimer with 2 long C- |
| | | term helices |
| retrorvirus capsid protein, | 5 helix | bundle |
| Nter core domain | | |
| influenza virus matrix | multihelical | 2 4-helix domains |
| protein M1 | | |
| (orbivirus, phytoreovirus, | multihelical | 3-helix bundle surrounded by |
| group A rotavirus) | | non-conserved helices |
| rhabdovirus | multihelical | 2 helical domains each with 1 |
| nucleoprotein-like | | buried helix |

ALL-BETA DOMAINS

| | | |
|---|---|---|
| nucleoplasmin-like/VP | beta-sheet sandwich of | 2 sheets, some with additional |
| (viral coat & capsid | 8 strands | 1-2 strands; jellyroll, forms 5- |
| proteins) | | fold and pseudo 6-fold |
| | | subassemblies |
| baculovirus p35 protein | 14 strands | 2 sheets, Greek key |
| coronavirus RNA-binding | 5 strands | coiled antiparallel 51324; |
| domain | | complex topology with |
| | | crossing loops |
| capsid top domain (bovine | 9 strands | 2 sheet sandwich; jellyroll, |
| rotavirus, bluetongue, | | forms trimers |
| rice dwarf, African horse | | |
| sickness virus) | | |

ALPHA + BETA (α + β) DOMAINS

| | | |
|---|---|---|
| coronavirus NSP8-like | a-b2-a-b4-a-b | bifurcated barrel-like beta |
| | | sheet |
| tombusvirus p19 core | b2-a-b2-a | antiparallel sheet 2134; 2 |
| protein vp19 | | layers: alpha/beta |
| rotavirus nps2 fragment, | 6 helices, 2 beta | |
| Nter domain | hairpins | |
| rna bacteriophage capsid | 6 strands-2 helices | meander of 6 strands followed |
| protein | | by 2 helices |

ALPHA + BETA (multidomain α + β) DOMAINS

| | | |
|---|---|---|
| reovirus inner layer core | numerous all-alpha | |
| protein c3 | regions, all-beta | |
| | domain near Cter | |
| L-A virus major coat | | large protein without apparent |
| protein | | domain division |
| major capsid protein VP5 | | large protein without apparent |
| | | domain division |

As a result of evolution each SCOP structure class has many member viruses. Because of the highly ordered packing of capsid proteins required to form a VLP, e.g. 60 capsid proteins for T=1 icosahedral viruses and 180 capsid proteins for T=3 icosahedral viruses, the capsid proteins of members within a structure class necessarily form VLPs with structural similarity. Members of the SCOP structure classes of interest, RNA bacteriophage capsid proteins and nucleoplasmin-like/VP (viral coat & capsid proteins), with publicly available atomic level crystal structures are provided in the Table below of SCOP subsets.

TABLE 5
SCOP Subsets
SCOP structure class/subclass/subclass members

ALL-BETA DOMAINS
nucleoplasmin-like/VP (viral coat & capsid proteins)
Positive stranded ssRNA viruses

| | |
|---|---|
| picornaviridae | human enterovirus B (coxsackieviruses B3 & A9; echoviruses 1 & |
| | 11) |
| | bovine enterovirus (bovine enterovirus VG-5-27) |
| | poliovirus (type 1 str Mahoney, type 2 str Lansing, type 3 str Sabin) |
| | rhinovirus (human rhinoviruses B 14, 16, A 1A, A 2, 3) |
| | aphthovirus (foot and mouth disease) |
| | theilovirus (theiler's murine encephalomyelitis str da) |
| | mengo encephaomyocarditis (mengovirus) |
| | swine vesicular disease (swine vesicular disease) |
| | insect picorna-like (cricket paralysis virus) |
| comoviridae | tobacco ringspot virus |
| | comovirus VP37 |
| | comovirus VP23 |
| | cowpea mosiac virus |
| | bean pod mottle virus |
| caliciviridae | Norwalk virus |
| nodaviridae-like | black beetle virus |
| | nodamura virus |
| | pariacoto virus |
| tetraviridae | nudaurelia capensis omega virus |
| bromoviridae | cucumber mosiac virus str fny |
| | tomato aspermy virus |
| | brome mosiac virus |
| | cowpea chlorotic mottle virus |
| tymoviridae | physalis mottle tymovirus |
| | desmodium yellow mottle tymovirus |
| | turnip yellow mosiac virus |
| tombusviridae | necrovirus (tobacco necrosis virus) |
| | tombusvirus (tomato bushy stunt virus) |
| | carmovirus (carnation mottle virus) |
| | sobemovirus |
| | sesbania mosaic virus |
| | souther bean mosiac virus str cowpea |
| | rice yellow mottle virus |
| | cocksfoot mottle virus |
| birnaviridae | birnavirus VP2 |
| | infectious bursal disease virus |

ssDNA viruses

| | |
|---|---|
| microviridae | phi-X174 |
| | G4 |
| | alpha3 |
| parvoviridae | feline panleukopenia virus str b |
| | canine parvovirus |
| | feline parvovirus |
| | porcine parvovirus |
| | murine minute virus str I |
| | human parvovirus b19 |
| | adeno-associated virus |
| | aav-2 |
| densiverinae- | galleria mellonella densovirus |

Group I dsDNA viruses VP (papovaviridae)

| | |
|---|---|
| papovaviridae | murine polymavirus str small placque |
| | simian virus 40 |
| | human papillomavirus L1 |

Group II dsDNA viruses

| | |
|---|---|
| p3 | bacteriophage prd1 |
| vp54 | paramecium bursaria chlorella virus 1 |
| adenovirus hexon | human adenoviruses type 5) |

Satellite viruses

| | |
|---|---|
| SPMV | satellite panicum mosiac virus |
| STMV | satellite tobacco mosiac virus |
| | satellite tobacco necrosis virus |

ALPHA + BETA (α/β) DOMAINS
RNA bacteriophage capsid protein
RNA bacteriophage capsid protein

| | |
|---|---|
| levivirus capsid proteins | MS2 |
| | FR |
| | GA |
| | PP7 |
| allolevirus capsid proteins | Qbeta |

### A. SCOP Classification RNA Bacteriophage Capsid Protein

Leviviridae and alloleviviridae capsid proteins belong to the RNA bacteriophage capsid protein class. Their domain is a meander of a 6-stranded beta-sheet followed by two alpha-helices. The latter are sometimes described as a long alpha-helix with a kink. In the assembled capsid the helices pack across the beta-sheet of neighboring capsid proteins. Representative structures of levi- and alloleviviridae capsid proteins deposited in the Protein Data Bank are provided in the Table 6 below.

TABLE 6
Representative, nonredundant Alpha + Beta, RNA bacteriophage
capsids with public domain structures classified by SCOP,
included in the RCSB and ViperDB. In each case the highest
elevated surface texture is a strand-turn- strand feature.

| | | | | | | | |
|---|---|---|---|---|---|---|---|
| | | | R- | | | | |
| | | reso- | factor | | | | outer |
| | PDB- | lution | and R- | chain | T- | No. of | diameter |
| Type | ID | (Å) | free | refined | number | subunits | (Å) |

levivirus

| | | | | | | | |
|---|---|---|---|---|---|---|---|
| MS2 | 1aq3 | 2.80 | 0.204 | 3*129aa | 3 | 180 | 288 |
| | | | | chains | | | |
| GA | 1gav | 3.40 | 0.279 | 45*129aa | 3 | 180 | 288 |
| | | | | chains | | | |
| FR | 1frs | 3.50 | 0.228 | 3*129aa | 3 | 180 | 286 |
| | | | and | chains | | | |
| | | | 0.236 | | | | |
| PP7 | 1dwn | 3.50 | 0.288 | 3*127aa | 3 | 180 | 286 |
| | | | and | chains | | | |
| | | | 0.292 | | | | |

allolevivirus

| | | | | | | | |
|---|---|---|---|---|---|---|---|
| Qbeta | 1qbe | 3.50 | 0.304 | 3*132aa | 3 | 180 | 294 |
| | | | | chains | | | |

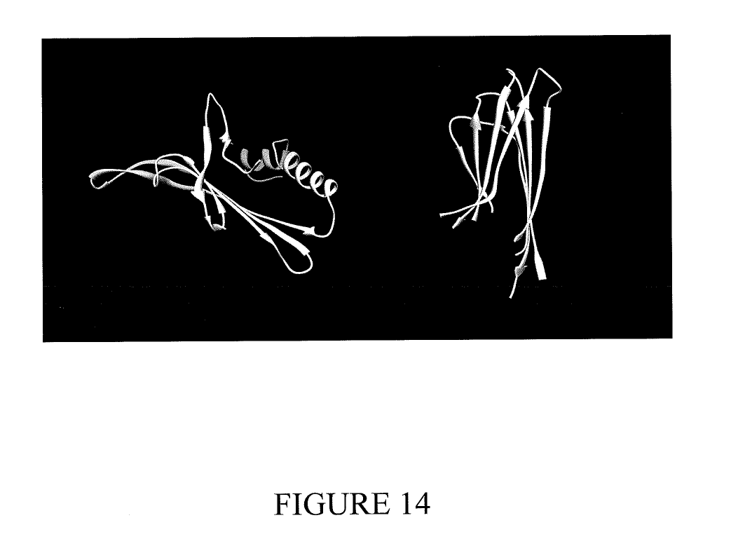

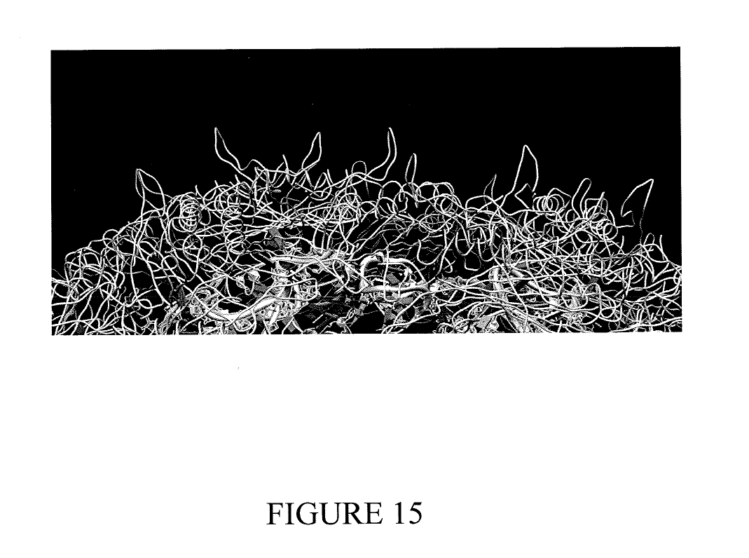

A backbone ribbon representation of the SCOP structure class is shown on the left of FIG. 14 (alpha+beta and all-beta common domain). A backbone ribbon diagram of part of the leviviridae MS2 capsid reconstructed from PDB-ID:1AQ3 is shown in FIG. 15 (1AQ3 levivirus MS2 alpha+beta T3 ribbon) wherein the capsid protein is shown in white while encapsulated fragments of RNA localized in the electron density are shown in color. Of note, the capsid surface is quite smooth and relatively featureless except for the long loop connecting the sheet and helix in the domain (FIG. 14, alpha+beta and all-beta common domain, left panel, pointing up). Comparable ribbon diagrams of the other entries in the Alpha+beta Table are virtually indistinguishable. This strand-turn-strand feature is the highest point of the exterior capsid topology.

### B. SCOP Classification Nucleoplasmin-Like/VP (Viral Coat & Capsid Proteins)

Positive stranded ssRNA viruses belonging to the comoviridae, caliciviridae, nodaviridae, tetraviridae, bromoviridae, tymoviridae, tombusviridae and birnaviridae; ssDNA viruses belonging to the microviridae, parvoviridae and densoviridae; group I dsDNA viruses belonging to the papovaviridae; coat protein S-type capsid proteins belonging to the group II dsDNA viruses and satellite viruses belong to the nucleoplasmin-like/VP (viral coat & capsid proteins) structure class contain domains comprising at least 8 beta-strands forming two beta sheets in a sandwich or jellyroll. Some subclasses contain one or two additional beta-strands in the sheets. Representative structures of positive stranded ssRNA viral capsid proteins deposited in the Protein Data Bank are provided in the Table below entitled “All-beta”). A backbone ribbon representation of the SCOP structure class is shown on the right of FIG. 14 (alpha+beta and all-beta common domain).

TABLE 7
Representative, nonredundant ALL BETA capsids with public domain structures classified by SCOP & included in ViperDB

| | | | | | | | | | |
|---|---|---|---|---|---|---|---|---|---|
| | | | | | | | | highest elevated | additional high |
| | | | | | | # | outer diameter | surface | elevation |
| pdb-id | resolution | R factor | R free | chain refined | T-number | subunits | (A) | texture | sites |

POSITIVE STRANDED ssRNA VIRUSES

| | | | | | | | | | | | | | |
|---|---|---|---|---|---|---|---|---|---|---|---|---|---|
| calciviridae-like VP | calicivirus | norwalk virus | lihm | 3.50 | 0.260 | | 3*530aa | 3 | 180 | 400 | 2 3strand | | |
| | | | | | | | chains | | | | perpendicular | | |
| | | | | | | | | | | | sheets | | |
| nodaviridae- | alphanodavirus | black beetle | 2bbv | 2.80 | 0.221 | | 3*363aa | 3 | 180 | 344 | long loop | second | |
| like VP | | virus | | | | | chains | | | | insertion in | sandwich | |
| | | | | | | | | | | | sandwich | | |
| | | nodamura | 1nov | 3.50 | 0.296 | | 3*355aa | 3 | 180 | 358 | long loop | | |
| | | virus | | | | | chains | | | | insertion in | | |
| | | | | | | | | | | | sandwich | | |
| | | pariacoto | 1f8v | 3.00 | 0.218 | 0.221 | 3*355aa | 3 | 180 | 350 | long loop | | |
| | | virus | | | | | chains | | | | insertion in | | |
| | | | | | | | | | | | sandwich | | |
| bromovirida- | cucomovirus | cucumber | 1f15 | 3.20 | 0.246 | | 3*218aa | 3 | 180 | 302 | sandwich | | |
| like VP | | mosiac virus, | | | | | chains | | | | | | |
| | | str fny | | | | | | | | | | | |
| | | tomato | 1laj | 3.40 | 0.218 | 0.228 | 3*217aa | 3 | 180 | 300 | sandwich | helical | |
| | | aspermy | | | | | chains | | | | | insertion in | |
| | | virus | | | | | | | | | | sandwich | |
| | | brome | 1js9 | 3.40 | 0.240 | 0.250 | 3*189aa | 3 | 180 | 284 | sandwich | helical | |
| | | mosiac virus | | | | | chains | | | | | insertion in | |
| | | | | | | | | | | | | sandwich | |
| | | cowpea | 1cwp | 3.20 | 0.310 | | 3*190aa | 3 | 180 | 288 | sandwich | helical | |
| | | chlorotic | | | | | chains | | | | | insertion in | |
| | | mottle virus | | | | | | | | | | sandwich | |
| tymoviridae- | tymovirus | physalis | 1e57 | 3.20 | 0.279 | 0.296 | 3*188aa | 3 | 180 | 316 | sandwich | | |
| like vp | | mottle virus | | | | | chains | | | | | | |
| | | desmodium | 1ddl | 2.70 | 0.152 | 0.159 | 3*188aa | 3 | 180 | 318 | sandwich | | |
| | | yellow mottle | | | | | chains | | | | | | |
| | | tymovirus | | | | | | | | | | | |
| | | turnip yellow | 1auy | 3.00 | 0.187 | 0.193 | 3*190aa | 3 | 180 | 316 | sandwich | | |
| | | mosiac virus | | | | | chains | | | | | | |
| tombusviridae-like VP | necrovirus | tobacco | 1c8n | 2.25 | 0.253 | 0.273 | 3*276aa | 3 | 180 | 318 | sandwich | helical | |
| | | mosiac virus | | | | | chains | | | | | insertion in | |
| | | | | | | | | | | | | sandwich | |
| | tombusvirus | tomato bushy | 2tbv | 2.90 | | | 3*387aa | 3 | 180 | 352 | sandwich | sandwich | sandwich |
| | | stunt virus | | | | | chains | | | | | at 6-fold | at 5- |
| | | | | | | | | | | | | symmetry | fold |
| | | | | | | | | | | | | point | symmetry |
| | | | | | | | | | | | | | point |
| | carmovirus | carnation | 1opo | 3.20 | 0.183 | | 3*348aa | 3 | 180 | 354 | sandwich | sandwich | sandwich |
| | | mottle virus | | | | | chains | | | | | at 6-fold | at 5- |
| | | | | | | | | | | | | symmetry | fold |
| | | | | | | | | | | | | point | symmetry |
| | | | | | | | | | | | | | point |
| | sobemovirus | sesbania | 1smv | 3.00 | 0.227 | | 3*266aa | 3 | 180 | 320 | sandwich | helical | |
| | | mosiac virus | | | | | chains | | | | | insertion in | |
| | | | | | | | | | | | | sandwich | |
| | | southern | 4sbv | 2.80 | 0.254 | | 3*260aa | 3 | 180 | 320 | sandwich | helical | |
| | | bean mosiac | | | | | chains | | | | | insertion in | |
| | | virus, | | | | | | | | | | sandwich | |
| | | cowpea str. | | | | | | | | | | | |
| | | rice yellow | 1f2n | 2.80 | 0.227 | 0.219 | 3*238aa | 3 | 180 | 318 | sandwich | helical | |
| | | mottle virus | | | | | chains | | | | | insertion in | |
| | | | | | | | | | | | | sandwich | |
| | | cocksfoot | 1ng0 | 2.70 | 0.281 | | 3*253aa | 3 | 180 | 320 | sandwich | helical | |
| | | mottle virus | | | | | chains | | | | | insertion in | |
| | | | | | | | | | | | | sandwich | |
| birnaviridae- | birnavirus | infectious | 2df7 | 2.80 | 0.168 | 0.215 | 20*458aa | 1 | 60 | 272 | sandwich | second | |
| like VP | VP2 | bursal | | | | | chains | | | | | sandwich | |
| | | disease virus | | | | | | | | | | at 3-fold | |
| | | | | | | | | | | | | symmetry | |
| | | | | | | | | | | | | points | |

ssDNA VIRSUSES

| | | | | | | | | | | | | |
|---|---|---|---|---|---|---|---|---|---|---|---|---|
| microviridae- | microvirus | bacteriophage | 2bpa | 3.00 | 0.209 | | 1*426aa | 1 | 60 | 342 | sandwich | 1 |
| like VP | | phi-X174 | | | | | chain, | | | | of spike | |
| | | | | | | | 1*175aa | | | | protein | |
| | | | | | | | chain | | | | | |
| | | bacteriophage | 1gff | 3.00 | 0.352 | | 1*426aa | 1 | 60 | 342 | sandwich | |
| | | g4 | | | | | chain, 1*177aa | | | | of spike | |
| | | | | | | | chain | | | | protein | |
| | | bacteriophage | 1m06 | 3.50 | 0.232 | 0.234 | 1*431aa | 1 | 60 | 342 | sandwich | |
| | | alpha3 | | | | | chain, 1*187aa | | | | of spike | |
| | | | | | | | chain | | | | protein | |
| parvoviridae- | parvovirus | feline | 1fpv | 3.30 | | | 1*584aa | 1 | 60 | 286 | sandwich | loops at a- |
| like VP | | panleukopenia | | | | | chain | | | | | fold |
| | | virus, str b | | | | | | | | | | symmetry |
| | | | | | | | | | | | | points |
| | | canine | 1c8d | 3.00 | 0.214 | | 1*584aa | 1 | 60 | 288 | sandwich | loops at 3- |
| | | parvovirus | | | | | chain | | | | | fold |
| | | | | | | | | | | | | symmetry |
| | | | | | | | | | | | | points |
| | | feline | 1c8g | 3.00 | 0.245 | | 1*584aa | 1 | 60 | 286 | sandwich | loops at 3- |
| | | parvovirus | | | | | chain | | | | | fold |
| | | | | | | | | | | | | symmetry |
| | | | | | | | | | | | | points |
| | | porcine | 1k3v | 3.50 | 0.283 | 0.283 | 1*579aa | 1 | 60 | 284 | sandwich | loops at 3- |
| | | parvovirus | | | | | chains | | | | | fold |
| | | | | | | | | | | | | symmetry |
| | | | | | | | | | | | | points |
| | | human | 1s58 | 3.50 | 0.313 | 0.316 | 1*554aa | 1 | 60 | 272 | sandwich | loops at 3- |
| | | parvovirus | | | | | chain | | | | | fold |
| | | B19 | | | | | | | | | | symmetry |
| | | | | | | | | | | | | points |
| | dependovirus | adeno- | 1lp3 | 3.00 | 0.338 | 0.342 | 1*519aa | 1 | 60 | 294 | sandwich | |
| | | associated | | | | | chain | | | | | |
| | | virus, aav-2 | | | | | | | | | | |
| densoviridae- | densovirus | galleria | 1dnv | 3.60 | 0.271 | | 1*437aa cain | 1 | 60 | 266 | sandwich | |
| like VP | | mellonella | | | | | | | | | | |
| | | densovirus | | | | | | | | | | |

GROUP I dsDNA VIRUSES

| | | | | | | | | | | |
|---|---|---|---|---|---|---|---|---|---|---|
| papillomavirus | human | 1dzl | 3.50 | 0.280 | 0.290 | 1*505aa chain | 1 | 60 | 320 | sandwich |
| L1 protein | papillomavirus | | | | | | | | | |
| | type 16 | | | | | | | | | |

SATELLITE VIRUSES

| | | | | | | | | | | | |
|---|---|---|---|---|---|---|---|---|---|---|---|
| satellite | SPMV coat | satellite | 1stm | 1.90 | 0.210 | | 5*157aa chains | 1 | 60 | 170 | sandwich |
| viruses | protein | panicum | | | | | | | | | |
| | | mosiac virus | | | | | | | | | |
| | STMV coat | satellite | 1a34 | 1.81 | 0.179 | 0.184 | 1*159aa chain | 1 | 60 | 176 | sandwich |
| | protein | tobacco | | | | | | | | | |
| | | mosaic virus | | | | | | | | | |
| | STNV coat | satellite | 2buk | 2.45 | 0.273 | | 1*196aa chain | 1 | 60 | 196 | sandwich |
| | protein | tobacco | | | | | | | | | |
| | | necrosis virus | | | | | | | | | |

Also included in the nucleoplasmin-like/VP (viral coat and capsid proteins) structure class are capsid proteins which are identified as sequence or structure homologs to any of the above capsids by employing sequence alignment and/or structure-anchored sequence alignment algorithms and methodologies well known and readily available to those of routine skill in the art. To make the identification, algorithms and methodologies can be applied to the full length sequences, or where appropriate, domain-wise. In the latter case, the domains would be as defined, for example, in the UniProt public domain database (Universal Protein Resource: The UniProt Consortium, (2012) Reorganizing the protein space at the Universal Protein Resource (UniProt) Nucleic Acids Res. 40: D71-D75, http://www.uniprot.org), SCOP or the Protein Data Bank. Optimized structure superpositions can easily be performed with programs like Chimera (Pettersen, E. F., Goddard, T. D., Huang, C.C., Couch, G. S., Greenblatt, D. M., Meng, E. C., and Ferrin, T. E. (2004) “UCSF Chimera-A Visualization System for Exploratory Research and Analysis.” J. Comput. Chem. 25:1605-1612; http://www.cgl.ucsf.edu/chimera) known to practitioners of the art.

The domain in the nucleoplasmin-like/VP (viral coat and capsid protein) structure class comprises 8 beta-strands forming two beta sheets in a sandwich or jellyroll. Capsid protein sequences within a family of virus can be identified and aligned, for example, with BLAST (threshold=10, Auto weighting array selection, no filtering, gaps allowed). Two or more families can be accurately aligned with respect to the optimal backbone overlay of model(s) of representative members of each family.

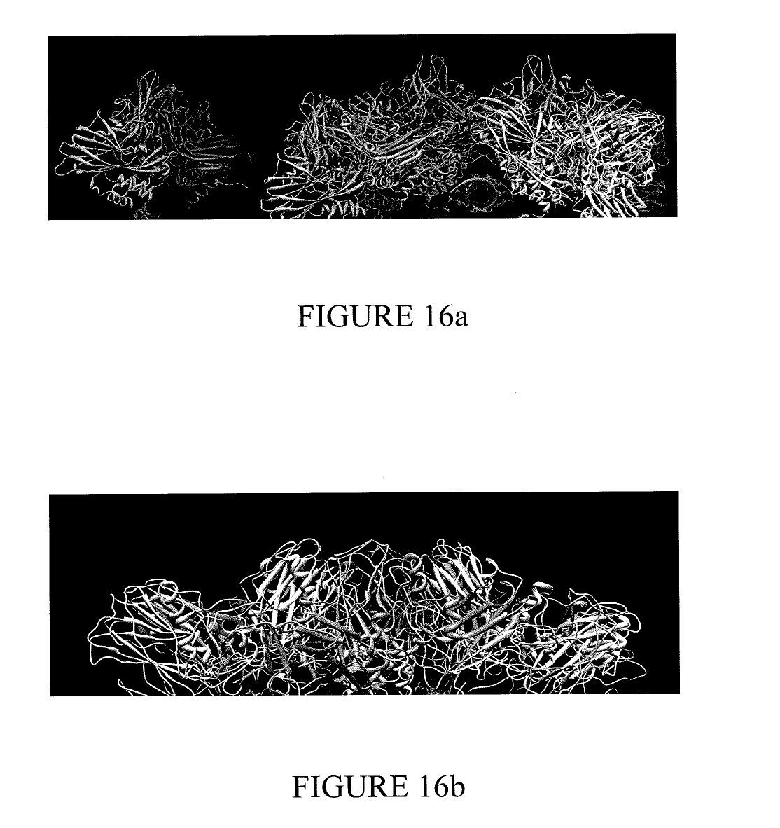

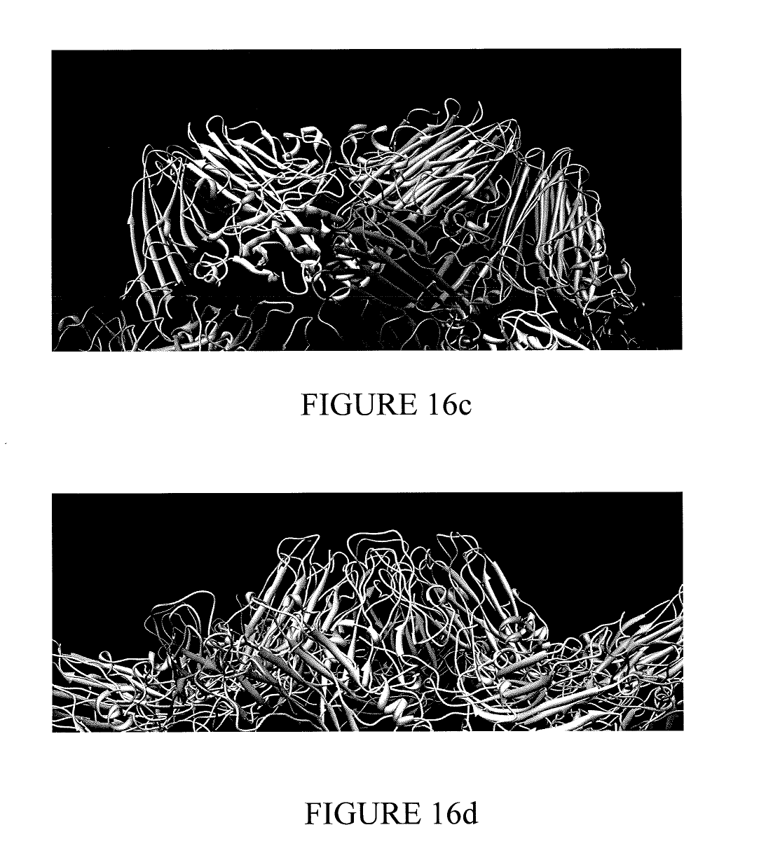

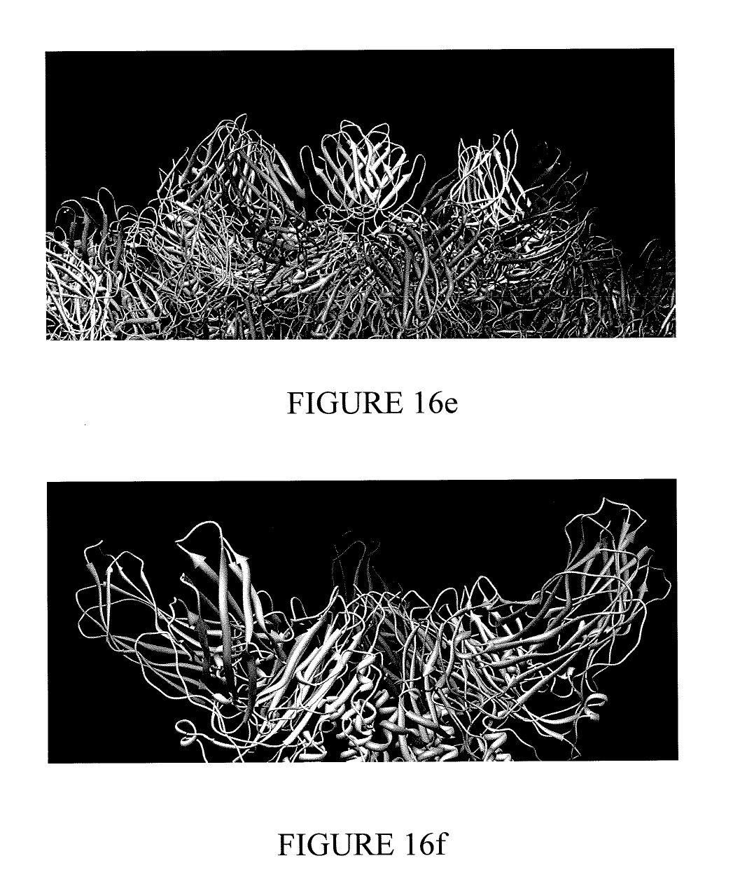

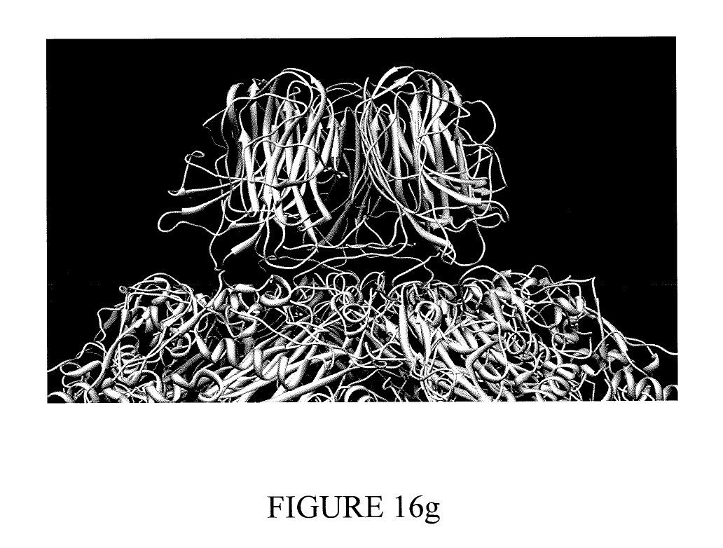

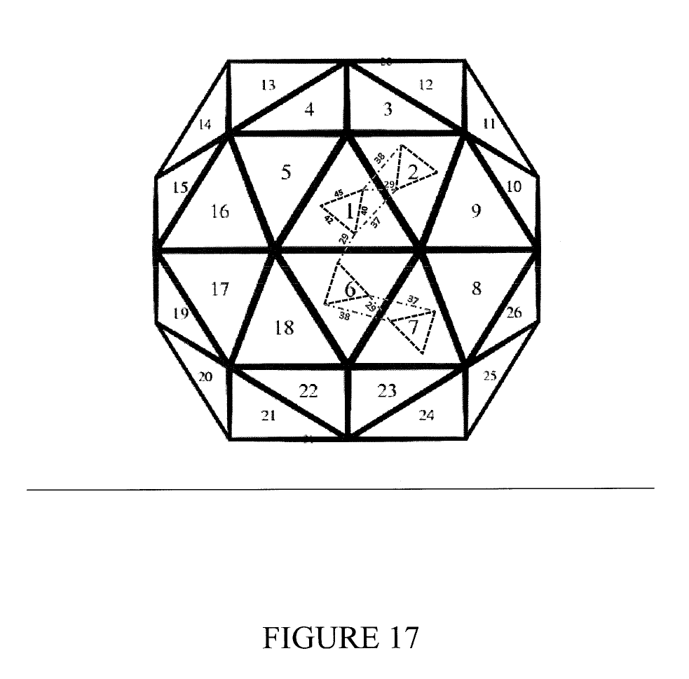

Even though the outer capsid surfaces of these viruses can appear quite different (see FIG. 13 sample textures of spherical capsids), the highest elevations on the capsid surface are formed by a very small set of secondary structures, typically the loops at the top of the SCOP domain as oriented on the left side of FIG. 14 (alpha+beta and all-beta common domain) or insertions in those loops. This can be illustrated for the inexpert eye with backbone ribbon diagrams of portions of capsid surfaces. Representative examples are provided in FIG. 16 a-g (2BBV alphanodavirus black beetle all beta T3 ribbon; 1LAJ bromovirus tomato aspermy virus all beta T3 ribbon; 2BUK STNV all beta T1 ribbon; 1E57 tymovirus physalis mottle virus all beta T3 ribbon; 2TBV tombusvirus tomato bushy stunt virus all beta T3 ribbon; 2DF7 infectious bursal disease virus all beta T1 ribbon; 2BPA bacteriaphage phi-X174 all beta T1 ribbon). These include T=1 and T=3 capsids (see All-beta Table). Ribbons are colored by asymmetric unit. This means that each coat protein in T=1 capsids is distinguished by a different shade. For T=3 capsids, the three nearest neighbor capsid proteins of the asymmetric unit share the same ribbon color.

III. Limiting Proteolysis of VLPs During Purification and Storage

The most likely locations on the VLP surface for productive binding of a hydrolase unencumbered by the hydrolase footprint is at the high topology points. At these points the hydrolase has a maximum number of approach angles to the capsid protein that allow the capsid protein to enter the active site deeply enough and with its backbone running in the proper direction for productive binding while avoiding steric collisions between the capsid surface and the rest of the hydrolase that push the hydrolase away from the capsid surface before proteolysis can occur. Deeper in the VLP texture productive approach angles of the hydrolase to the capsid are more limited and the chance a hydrolase-capsid protein encounter will result in backbone cleavage is considerably smaller. Further, the effective hydrolase footprint can become quite a bit larger if more of the hydrolase is required to descend into texture. Mutational alteration of the specific structure of these regions is most likely to affect the protein hydrolysis properties of the structure.

Once the location of the preferred hydrolase binding motifs on the surface of the VLP have been identified the possibility of a productive encounter and an estimate of successful approach angles can be done a number of ways. For example, approach angles can be determined by: 1) simple geometry (as in section A below); 2) loading crystal structures or homology models of good quality into commonly used modeling programs and attempting a static docking of the capsid protein into the hydrolase active site using software like Chimera; 3) attempting to dynamically dock the capsid protein and hydrolase protein using molecular dynamics software, for example CHARMM (B. R. Brooks, C. L. Brooks III, A. D. Mackerell Jr., L. Nilsson, R. J. Petrella, B. Roux, Y. Won, G. Archontis, C. Bartels, S. Boresch, A. Caflisch, L. Caves, Q. Cui, A. R. Dinner, M. Feig, S. Fischer, J. Gao, M. Hodoscek, W. Im, K. Kuczera, T. Lazaridis, J. Ma, V. Ovchinnikov, E. Paci, R. W. Pastor, C. B. Post, J. Z. Pu, M. Schaefer, B. Tidor, R. M. Venable, H. L. Woodcock, X. Wu, W. Yang, D. M. York, & M. Karplus, (2009) J Comput Chem 30, 1545-1614) and mimicking the process of induced fit during a successful encounter; or 4) estimating enzyme binding requirements and matching them against sequence/structure data using homology modeling arguments familiar to experts (as described in section B below).

Conversely, proteolysis can be limited by removing preferred binding motifs from locations that can successfully bind to hydrolase via the substitution, insertion or deletion of residues and in principle, final yields from a VLP manufacturing process can be increased.

One method for identifying or characterizing capsid proteins that are resistant to hydrolases as described herein is to quantify the limitations on surface loops of the protein in terms of length of projection from the surface, based on defined points A and B with reference to a 3-D molecular model of a given capsid protein as obtained by or derived from X-Ray diffraction, wherein:
Point B is the average position of 300 backbone atoms (not including oxygen or hydrogen) belonging to the capsid that meet the following two conditions: 1) the atoms don't belong to the loop; and 2) the atoms are closer to point A than any other backbone atom in the capsid.
Point A is the average position of the backbone atom in the loop, such atom being the one located the farthest away from point B.
   Given the foregoing, the distance between point A and Point B should be no more than 13-15 Angstroms, preferably less than 10-12 Angstroms, and more preferably less than 6-9 Angstroms.

Calculation protocol for distance between A and B, using the 3-D structure obtained using X-Ray diffraction:

a) Picking an amino acid in a loop and any backbone atom in such amino acid.

b) Fixing point A as average position of the atom picked in (a).

c) Picking the 300 backbone atoms closest to the atom picked in (a) which don't belong to the loop.

d) Fixing point B as average position of the 300 atoms picked in (c).

e) Calculating distance between Point A and B.

f) Repeating (a) through (e) for every backbone atom in the chosen loop.

g) Largest distance obtained in (f) should be no more than 13-15 Angstroms, preferably less than 10-12 Angstroms, and more preferably less than 6-9 Angstroms.

### A. Estimation of Hydrolysis of the Leviviridae MS2 VLP by Simple Geometry

The only high points of topology with respect to the outer surface of the assembled capsid are the loops shown in FIGS. 14 and 15 (alpha+beta and all-beta common domain and 1AQ3 levivirus MS2 alpha+beta T3 ribbon), comprised by residues 7-20 and extending 10-12 Angstroms above the bulk of the VLP, a sufficient distance to be able to bind in the hydrolase active site. Because the nearest neighbor loops are at least 29 Angstroms away (FIG. 17), the interaction between hydrolase and capsid protein is unencumbered by the hydrolase footprint. However, the outer portion of the loop does not contain any of the hydrolase binding motifs given in Table 2 so MS2 is expected to be stable to hydrolysis by these enzymes, and is in fact, quite stable to hydrolysis as shown herein above.

Conversely, if the center of the loop is comprised one or more of the binding motifs given in Table 2, the capsid would be expected to be susceptible to proteolysis by the corresponding EC 3.4 hydrolase(s). In this case, the capsid sequence would be discarded in favor of one more hydrolase-resistant one without EC 3.4 hydrolase binding motifs in the center of this loop or, alternatively, the motifs could be bioengineered away by replacing or deleting the motif residues. A practitioner of the art would take care to use standard methods to avoid bioengineering changes that could disturb the local fold substantially and possibly distorting the assembled capsid.

This approach can be applied to any viral capsid protein for which one or more x-ray structures of good quality are available, either of the viral capsid or capsid protein(s) of interest, a homologous viral capsid or capsid protein(s) as identified by an algorithm familiar to experts in the field, i.e. BLAST, or a viral capsid or capsid protein(s) related via structure-anchored alignment.

### B. Estimation of Hydrolysis by Analyzing Substrate, Inhibitor, Analog or Modeled Compound Docking to a Hydrolase

Public domain Cartesian coordinate sets of representative x-ray structures solved at high resolution and of good quality are available for the EC3.4 hydrolases likely to be used in commercial processes. These can be critically and quantitatively examined to determine the characteristics of local folds which enhance susceptibility to hydrolysis of a target protein by the hydrolase under examination, particularly the sites in the natively folded protein with the highest susceptibility to hydrolysis.

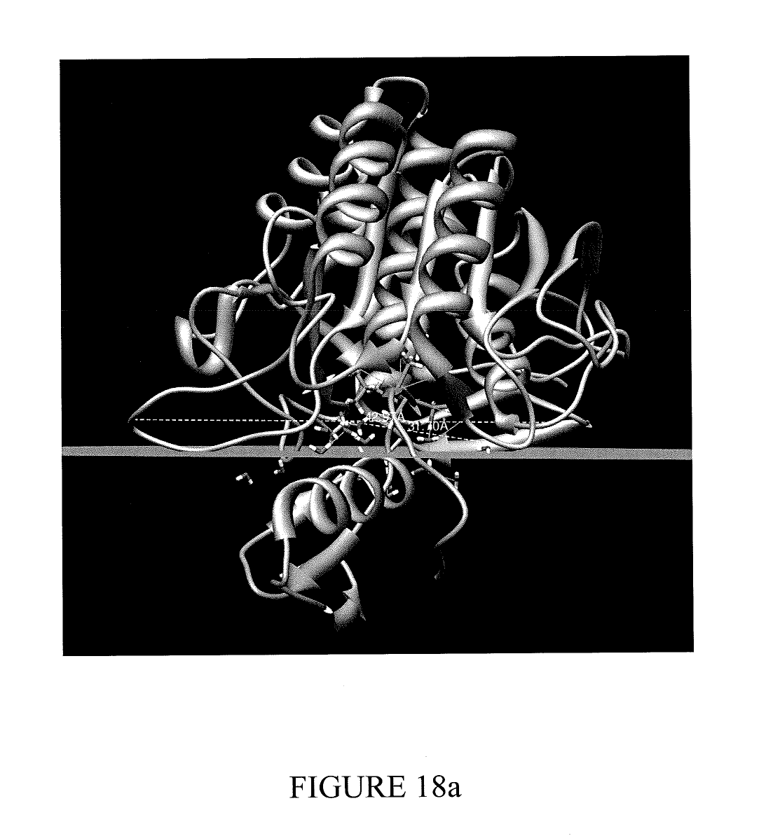

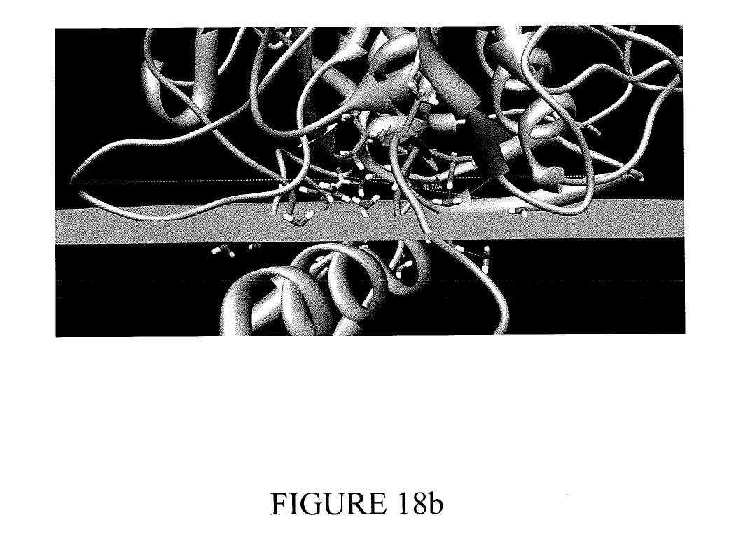

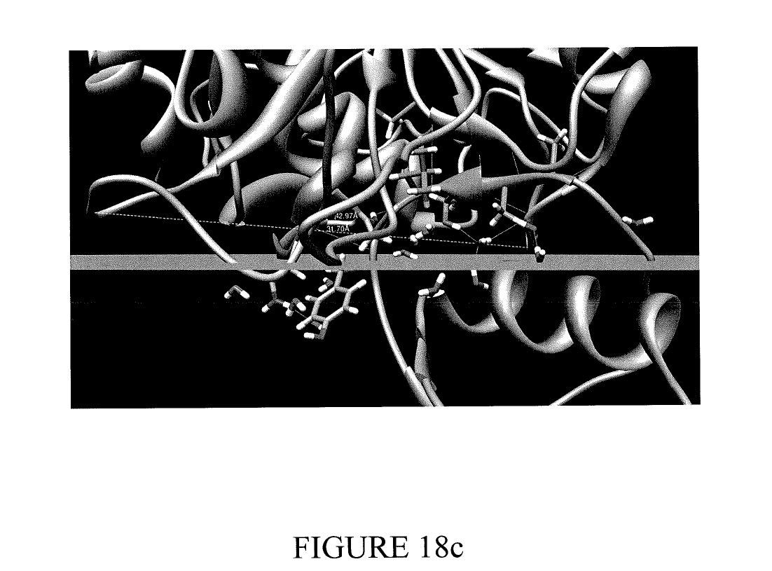

A schematic representation of a molecule of subtilisin A from Bacillus licheniformis (gray ribbon) complexed with the kazal-domain protein OKTYK3 (medium blue ribbon) from PDB-ID:1YU6 is provided in FIGS. 18 a-c. The subtilisin targets the peptide bond bound across its active site, here formed by residues Asp 32, His 64 and Ser 221 for cleavage, while substrate binding pockets formed from spatially proximate subtilisin residues interact with substrate residues adjacent to the cleavage site in the linear sequence of the target protein to constrain the local target protein backbone in a conformation most energetically favorable for a productive cleavage event. These substrate residues can be identified in a manner independent of the actual substrate residue identity by locating hydrogen bonds formed between the backbone of these residues and the hydrolase, shown in Table 8 for PDB-ID:1YU6. The Calpha atoms of these substrate residues participating in these hydrogen bonds to hydrolase, 15-18 in PDB-ID:1YU6, define the x-axis described previously in this example. The y- and z-axes are located by rotating the complex around the x-axis until well-defined hydrolase backbone spatially proximate to the binding cleft extends along the (−)z-axis the same distance all around the hydrolase. This determines the location of the x-y plane and establishes a universal protocol for comparing the approach of a hydrolase to a viral capsid surface loop with a potentially high probability of cleavage. In FIGS. 18 a-c the x-y plane is shown translated to the bottom of the hydrolase. The depth of capsid surface loop incursion into the hydrolase binding cleft required for a productive cleavage event can be estimated by measuring the perpendicular distance of the Calpha atoms used to determine the x-axis from the translated plane, shown for PDB-ID:1YU6 in Table 9.

The number of substrate Calpha atoms used to define the x-axis is the number of substrate residues required to bind to the hydrolase in order to achieve the most energetically favorable (most probable) cleavage, e.g. 4 residues for the subtilisin. The average distance between residues in an extended conformation, e.g. antiparallel beta-sheet, is 3.2 Angstroms. Therefore, the distance from the translated x-y plane and the N-terminal Calpha of this set divided by 3.2 Angstroms and rounded to the closest integer is the minimal number of loop residues N-terminal to the bound segment required for productive binding. In the case of subtilisin, this is calculated as 6.5 Angstroms/3.2 Angstroms=2.03, or about 2 residues. Similarly, the distance from the translated x-y plane and the C-terminal Calpha of this set divided by 3.2 Angstroms and rounded to the closest integer is the minimal number of loop residues C-terminal to the bound segment required for productive binding is calculated as 4.4 Angstroms/3.2 Angstroms=1.37, or about 2 residues. Consequently, a surface loop candidate for cleavage must be at least as long as the number of N-terminal and C-terminal residues required for binding to the hydrolase, e.g. 2+2+4=8 residues for subtilisin. OMTYK3 residues 17 and 18 lie within the active site, so cleavage of the peptide bond between residues 17 and 18 is anticipated. This corresponds to residues 5 and 6 in minimal length segment and from Table 2 subtilisin A has a single motif preference at position Xaa. Therefore, for high probability of cleavage by subtilisin A, a capsid surface loop must have at least 8 residues which are likely to rise into solvent above the surface of the capsid exterior and a residue with a large, uncharged sidechain (Table 2) at at least the sixth position or greater from the N-terminal end of the loop and simultaneously at at least the third position or greater from the C-terminal end of the loop. Any portion of viral capsid protein sequence meeting these criteria for surface exposure, loop length and motif position within the loop is likely to experience a productive cleavage event in the presence of subtilisin A. Since MS2 loop 7-20 does not meet these criteria, MS2 is expected to be resistant to subtilisin A. Moreover, the hydrolase resistant capsids will meet these criteria for multiple EC3.4 hydrolases.

If the EC3.4 hydrolase under consideration exists under the proteolytic conditions described herein as a biological unit comprised of more than one copy of the hydrolase or closely associated covalently or noncovalently with other proteins or moieties, the entire biological unit must be considered in the analysis.

Alternatively, the hydrolase complex(es) for analysis could be produced by molecular mechanics, molecular dynamics, Monte Carlo, QM/MM, homology modeling, de novo modeling, other experimental, theoretical or computational data and data manipulation techniques or some combination thereof familiar to practitioners of the art. Surface loops of highest susceptibility to hydrolase attack, can also be identified as segments in a refined x-ray structure of high resolution and good quality containing several residues with backbone atoms which are undefined in the electron density maps or are characterized by atomic B-factors above the average B-factor for the protein, especially B-factors exceeding preferably more than 1.5*Bavg(protein), more preferably more than 2.0*Bavg(protein) and most preferably more than 2.5*Bavg(protein).

If structure information for a target capsid coat protein is unavailable, capsid susceptibility to EC3.4 hydrolases can be estimated using this protocol by analogy to the known structures of highly homologous capsid proteins or capsid proteins associated through structure-anchored alignments.

TABLE 8
Relevant hydrogen bonds (D-H . . . A) formed between subtilisin
binding cleft residues (chain A) and the backbone of bound protein
domain OKTYK3 (chain C) in the complex PDB-ID: 1YU6

| | | | D A | |
|---|---|---|---|---|
| | | | distance | (D-H A) |
| donor | acceptor | hydrogen | (\acute{Å}) | (deg) |
| GLY 127.A N | CYS 16.C O | GLY 127.A H | 2.950 | 1.983 |
| ASN 155.A | LEU 18.C O | ASN 155.A | 2.712 | 1.731 |
| ND2 | | HD22 | | |
| ALA 15.C N | GLY 102.A O | ALA 15.C H | 3.065 | 2.131 |
| CYS 16.C N | GLY 127.A O | CYS 16.C H | 3.099 | 2.141 |
| THR 17.C N | GLY 100.A O | THR 17.C H | 3.102 | 2.119 |
| LEU 18.C N | SER 125.A O | LEU 18.C H | 3.296 | 2.398 |
| TYR 20.C N | ASN 218.A O | TYR 20.C H | 2.828 | 1.829 |

TABLE 9
Distances of Calpha atoms defining the x-axis
from the translated x-y plane of PDB-ID: 1YU6

| Calpha | Perpendicular distance |
|---|---|
| atom | to plane (\acute{Å}) |
| 15.C CA | 6.5 |
| 16.C CA | 4.3 |
| 17.C CA | 4.7 |
| 18.C CA | 4.4 |

1-20. (canceled)

21. A virus-like particle (VLP) comprising a capsid enclosing at least one heterologous cargo molecule and a packing sequence, wherein the capsid is resistant to hydrolysis catalyzed by a category EC 3.4 peptide bond hydrolase.

22. The VLP of claim 1, wherein the capsid comprises capsid protein having a surface structure wherein any surface loops lack enough residues to satisfy peptide bond hydrolase-VLP binding requirements.

23. The VLP of claim 2, wherein the capsid comprises capsid protein having a surface structure wherein any surface loops have a length of no more than 13-15 Angstroms, preferably less than 10-12 Angstroms, and more preferably less than 6-9 Angstroms.

24. The VLP of claim 1, wherein the capsid comprises capsid protein having a surface structure wherein any surface loops possess enough residues to satisfy peptide bond hydrolase-VLP binding requirements but do not possess the peptide bond hydrolase enzyme preferred binding motifs at the required residue positions within such loops.

25. The VLP of claim 1, wherein the category EC 3.4 peptide bond hydrolase is selected from the group consisting of peptidase K, pepsin A, papain, steptogrisin A, streptogrisin B, subtilisin and protease from Bacillus licheniformis.

26. The VLP of claim 1, wherein the capsid is selected from the capsid proteins listed in Table 5 and homologs thereof.

27. The VLP according to claim 1, wherein the capsid protein has a three dimensional structure comprising a meander of a 6-stranded beta-sheet followed by two alpha-helices.

28. The VLP according to claim 1, wherein the capsid protein has a three dimensional structure comprising two beta sheets comprising at least 8 beta strands, the two beta sheets forming a sandwich or jellyroll.

29. The VLP according to claim 1, wherein the heterologous cargo molecule comprises an oligonucleotide.

30. The VLP according to claim 1, wherein the heterologous cargo molecule comprises a peptide.

31. A composition comprising: a plurality of the VLPs of claim 1 and one or more cell lysis products present in an amount of less than 4 grams for every 100 grams of capsid present in the composition, wherein the cell lysis products are selected from proteins, polypeptides, peptides and any combination thereof.

32. The composition according to claim 11, wherein the capsid comprises capsid protein selected from the capsid proteins listed in Table 5 and homologs thereof.

33. The composition according to claim 11, wherein the capsid comprises capsid protein with a three dimensional structure comprising a meander of a 6-stranded beta-sheet followed by two alpha-helices.

34. The composition according to claim 11, wherein the capsid comprises capsid protein with a three dimensional structure comprising two beta sheets comprising at least 8 beta strands, the two beta sheets forming a sandwich or jellyroll.

35. A method to purify VLPs of claim 1, the method comprising: subjecting a plurality of the VLPs obtained from a whole cell lysate to hydrolysis using a category EC 3.4 peptide bond hydrolase, for a time and under conditions sufficient for at least 60, at least 70, at least 80, or at least 90 of every 100 individual polypeptides present with the capsids are cleaved, while at least 60, at least 70, at least 80, or at least 90 of every 100 capsids present before such hydrolysis remain uncleaved after such hydrolysis, wherein the polypeptides are cell lysis products not enclosed in the capsids, and wherein the viral capsids comprise a capsid protein having a surface structure wherein any surface loops have a length of no more than 13-15 Angstroms, preferably less than 10-12 Angstroms, and more preferably less than 6-9 Angstroms.

36. The method according to claim 15, wherein the category EC 3.4 peptide bond hydrolase is selected from the group consisting of peptidase K, pepsin A, papain, steptogrisin A, streptogrisin B, subtilisin and protease from Bacillus licheniformis.

37. The method according to claim 15, further comprising purification of the capsids following hydrolysis, wherein purification includes at least one of a liquid-liquid extraction step, a crystallization step, a fractional precipitation step or an ultrafiltration step.

38. A method to purify VLPs of claim 1, the method comprising: subjecting a plurality of the capsids obtained from a whole cell lysate to hydrolysis using a category EC 3.4 peptide bond hydrolase, for a time and under conditions sufficient for at least 60, at least 70, at least 80, or at least 90 of every 100 individual polypeptides present with the capsids are cleaved, while at least 60, at least 70, at least 80, or at least 90 of every 100 capsids present before such hydrolysis remain uncleaved after such hydrolysis, wherein the polypeptides are cell lysis products not enclosed in the capsids, and wherein the viral capsids comprise a capsid protein selected from the capsid proteins listed in Table 5 and homologs thereof

39. The method according to claim 18, wherein the category EC 3.4 peptide bond hydrolase is selected from the group consisting of peptidase K, pepsin A, papain, steptogrisin A, streptogrisin B, subtilisin and protease from Bacillus licheniformis.

40. The method according to claim 18, further comprising purification of the capsids following hydrolysis, wherein purification includes at least one of a liquid-liquid extraction step, a crystallization step, a fractional precipitation step or an ultrafiltration step.


SEQUENCE LISTING (46 sequences)

SEQ ID NO: 1 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 2 - protein, 129 aa, Enterobacteria phage MS2
ASNFTQFVLVDNGGTGDVTVAPSNFANGVAEWISSNSRSQAYKVTCSVRQSSAQNRKYSIKVEVPKVATQTVGGVELPVAAWRSYLNMELTIPIFATNSDCELIVKAMQGLLKDGNPIPSAIAANSGIY

SEQ ID NO: 3 - protein, 129 aa, Enterobacteria phage GA
ATLRSFVLVDNGGTGNVTVVPVSNANGVAEWLSNNSRSQAYRVTASYRASGADKRKYTIKLEVPKIVTQVVNGVELPGSAWKAYASIDLTIPIFAATDDVTVISKSLAGLFKVGNPIAEAISSQSGFYA

SEQ ID NO: 4 - protein, 129 aa, Enterobacteria phage FR
ASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 257 aa, Enterobacteria phage MS2
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYANFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 6 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 7 - protein, 120 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIP

SEQ ID NO: 8 - protein, 130 aa, Enterobacteria phage MS2
MASNFTQFVLVDNGGTGDVTVAPSNFANGVAEWISSNSRSQAYKVTCSVRQSSAQNRKYTIKVELPKVATQTVGGVELPVAAWRSYLNMELTIPIFATNSDCELIVKAMQGLLKDGNPIPSAIAANSGIY

SEQ ID NO: 9 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNVELTIPIFATNSDCELIVKAMQGLLKDGNPIPSAIAANSGIY

SEQ ID NO: 10 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNPDCELIVKAMQGLLKDGNPIPSAIAANSGIY

SEQ ID NO: 11 - protein, 130 aa, Enterobacteria phage MS2
MASNFTQFVLVDNGGTGDVAVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 12 - protein, 130 aa, Enterobacteria phage MS2
  misc_feature (50)..(50): Xaa can be any naturally occurring amino acid
MASNFTQFVLVDNGGTGDVTVAPSNFANGVAEWISSNSRSQAYKVTCSVXQSSAQNRKYTIKVEVPKVATQTVGGVELPVAAWRSYLNMELTIPIFATNSDCELIVKAMQGLLKDGNPIPSAIAANSGIY

SEQ ID NO: 13 - protein, 130 aa, Enterobacteria phage MS2
  misc_feature (7)..(7): Xaa can be any naturally occurring amino acid
MASNFTXFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 14 - protein, 130 aa, Enterobacteria phage BO1
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGPLKDGNPIPSAIAANSGIY

SEQ ID NO: 15 - protein, 130 aa, Enterobacteria phage MS2
MASNFTQFVLVDNDGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 16 - protein, 130 aa, Enterobacteria phage MS2
  misc_feature (18)..(18): Xaa can be any naturally occurring amino acid
MASNFTQFVLVDNGGTGXVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 17 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNLELTIPIFATNPDCELIVKAMQGLLKDGNPIPSAIAANSGIY

SEQ ID NO: 18 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPISSAIAANSGIY

SEQ ID NO: 19 - protein, 130 aa, Enterobacteria phage MS2
  misc_feature (104)..(104): Xaa can be any naturally occurring amino acid
MASNFTQFVLVDNGGTGDVTVAPSNFANGVAEWISSNSRSQAYKVTCSVRQSSAQNRKYTIKVEVPKVATQTVGGVELPVAAWRSYLNVELTIPIFATNSDCEXIVKAMQGLLKDGNPIPSAIAANSGIY

SEQ ID NO: 20 - protein, 130 aa, Enterobacteria phage MS12
  misc_feature (22)..(22): Xaa can be any naturally occurring amino acid
MASNFTQFVLVDNGGTGDVTVXPSNFANGVAEWISSNSRSQAYKVTCSVRQSSAQNRKYTIKVEVPKVATQTVGGVELPVAAWRSYLNMELTIPIFATNSDCALIVKAMQGLLKDGNPIPSAIAANSGIY

SEQ ID NO: 21 - protein, 130 aa, Enterobacteria phage MS2
  misc_feature (130)..(130): Xaa can be any naturally occurring amino acid
MASNFTQFVLVDNGGTGDVTVAPSNFANGVAEWISSNSRSQAYKVTCSVRQSSAQNRKYTIKVEVPKVATQTVGGVELPVAAWRSYLNVELTIPIFATNSDCELIVKAMQGLLKDGNPIPSAIAANSGIX

SEQ ID NO: 22 - protein, 130 aa, Enterobacteria phage MS2; identical to SEQ ID NO: 21
  misc_feature (130)..(130): Xaa can be any naturally occurring amino acid

SEQ ID NO: 23 - protein, 130 aa, Enterobacteria phage MS2
MASNFTQFVLVDNGGTGDVTVAPSNFANGVAEWISSNSRSQAYKVTCSVRQSSAQNRKYTIKVEVPKVATQTVGGVQLPVAAWRSYLNMELTIPIFATNDDCALIVKAMQGLLKDGNPIPSAIAANSGIY

SEQ ID NO: 24 - protein, 130 aa, Enterobacteria phage MS2; identical to SEQ ID NO: 23

SEQ ID NO: 25 - protein, 127 aa, Enterobacteria phage MS2
NFTQFVLVDNGGTGDVTVAPSNFANGVAEWISSNSRSQAYKVTCSVRQSSAQNRKYTIKVEVPKVATQTVGGVELPVAAWRSYLNVELTIPIFATNSDCELIVKAMQGLLKDGNPIPSAIAANSGIY

SEQ ID NO: 26 - protein, 129 aa, Enterobacteria phage ZR
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 27 - protein, 129 aa, Enterobacteria phage R17; identical to SEQ ID NO: 26

SEQ ID NO: 28 - protein, 130 aa, Enterobacteria phage MS2; identical to SEQ ID NO: 1

SEQ ID NO: 29 - protein, 130 aa, Enterobacteria phage MS2; identical to SEQ ID NO: 1

SEQ ID NO: 30 - protein, 130 aa, Enterobacteria phage MS2; identical to SEQ ID NO: 1

SEQ ID NO: 31 - protein, 130 aa, Enterobacteria phage ZR; identical to SEQ ID NO: 1

SEQ ID NO: 32 - protein, 130 aa, Enterobacteria phage JP501
MASNFTEFVLVDNGETGNVTVAPSNFANGVAEWISSDSRSQAYKVTCSVRQSSAQNRKYTIKVAVPKVATQTVGGVELPVAAWRSYLNMELTIPIFATNSDCALIVKAMQGLLKDGNPIPSAIAANSGIY

SEQ ID NO: 33 - protein, 129 aa, Enterobacteria phage F2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 34 - protein, 130 aa, Enterobacteria phage JP34
MATLRSFVLVDNGGTGDVTVVPVSNANGVAEWLSNNSRSQAYRVTASYRASGADKRKYTIKLEVPKIVTQVVNGVELPVSAWKAYASIDLTIPIFAATDDVTVISKSLAGLFKVGNPIADAISSQSGFYA

SEQ ID NO: 35 - protein, 130 aa, Enterobacteria phage SD
MATLRSFVLVDNGGTGNVTVVPVSNANGVAEWLSNNSRSQAYRVTASYRASGADKRKYTIKLEVPKIVTQVVNGVELPISAWKAYASIDLTIPIFAATDDVTTISKSLAGLFKVGNPIADAISSQSGFYA

SEQ ID NO: 36 - protein, 130 aa, Enterobacteria phage JP500; identical to SEQ ID NO: 34

SEQ ID NO: 37 - protein, 130 aa, Enterobacteria phage KU1
MATLRSFVLVDNGGTGNVTVVPVSNANGVAEWLSNNSRSQAYRVTASYRASGADKRKYTIKLEVPKIVTQSVNGVELPVSAWKAFASIDLTIPIFAATDDVTLISKSLAGLFKIGNPVADAISSQSGFYA

SEQ ID NO: 38 - protein, 130 aa, Enterobacteria phage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 39 - protein, 130 aa, Enterobacteria phage BZ13
MATLRSFVLVDNGGTGNVTVVPVSNANGVAEWLSNNSRSQAYRVTASYRASGADKRKYTIKLEVPKIVTQVVNGVELPVSAWKAYASIDLTIPIFAATDDVTVISKSLAGLFKVGNPIAEAISSQSGFYA

SEQ ID NO: 40 - protein, 130 aa, Enterobacteria phage BZ13
MATLRSFVLVDNGGTGNVTVVPVSNANGVAEWLSNNSRSQAYRVTASYRASGADKRKYTIKLEVPKIVTQTVNGVELPVSAWKAYASIDLTIPIFAATDDVTLISKSLAGLFKIGNPVADAISSQSGFYA

SEQ ID NO: 41 - protein, 130 aa, Enterobacteria phage BZ13
MATLRSFVLVDNGGTGNVTVVPVSNANGVAEWLSNNSRSQAYRVTASYRASGADKRKYTIKLEVPKIVTQVVNGVELPVSAWKAYASIDLTIPIFAATDDVTVISKSLAGLFKVGDPIADAISSQSGFYA

SEQ ID NO: 42 - protein, 130 aa, Enterobacteria phage TH1
MATLRSFVLVDNGGTGNVTVVPVSNANGVAEWLSNNSRSQAYRVTASYRASGADKRKYTIKLEVPKIVTQVVNGVELPVSAWKAYASIDLTIPIFAATDDVTVISKSLAGLFKVGNPIADAISSQSGFYA

SEQ ID NO: 43 - protein, 130 aa, Enterobacteria phage T12; identical to SEQ ID NO: 42

SEQ ID NO: 44 - protein, 130 aa, Enterobacteria phage FR
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 45 - protein, 130 aa, Enterobacteria phage R17; identical to SEQ ID NO: 1

SEQ ID NO: 46 - protein, 132 aa, Enterobacteria phage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY